(19) United States
(12) Patent Application Publication
(10) Pub. No.: US 2021/0079049 A1
(43) Pub. Date: Mar. 18, 2021

(54) Engineered Biofilms

(71) Applicant: ShanghaiTech University, Shanghai (CN)

(72) Inventors: Chao Zhong, Shanghai (CN); Chen Zhang, Shanghai (CN); Jiaofang Huang, Shanghai (CN); Jicong Zhang, Shanghai (CN)

(73) Assignee: ShanghaiTech University, Shanghai (CN)

(21) Appl. No.: 17/106,105

(22) Filed: Nov. 28, 2020

Related U.S. Application Data
(63) Continuation of application No. PCT/CN2019/088492, filed on May 27, 2019.

(30) Foreign Application Priority Data
May 28, 2018 (CN) CN/201810519230.7
Dec. 19, 2018 (CN) PCT/CN2018/121947

(51) Int. Cl. C07K 14/32; C07K 14/435; C07K 14/245; C07K 14/21; C07K 14/195; C09J 7/10; C09J 11/08; C09J 189/00; C09J 105/00

(52) U.S. Cl. CPC C07K 14/32; C07K 14/43504; C07K 14/245; C07K 14/21; C07K 14/195; C09J 7/10; C09J 2400/20; C09J 189/00; C09J 105/00; C07K 2319/00; C09J 2405/00; C09J 2489/00; C09J 11/08

(57) ABSTRACT

Engineered living glues made by bacteria biofilms integrate natural marine adhesive proteins and electrostatic interactions into bacterial biofilm components such as amyloid protein TasA, surface layer protein BslA, and exopolysaccharides.

### INTRODUCTION

Underwater adhesives are highly demanded advanced materials for many technological and biomedical applications in wet or high-moisture settings [1, 2]. In biomedicine, biocompatible adhesive hydrogels have found wide use for bonding tissues and filling skin loss wounds, among many other applications [3-5]. Further, adhesives with a large variety of configurable structures and properties have been widely explored in other industries, for example in electronics (e.g., ion-exchangeable battery separators, conductive and stretchable electronic skins, etc.) [6, 7]. The development of adhesives that work in these contexts is challenging, as instantaneous and robust adhesion at surfaces is required to achieve desired outcomes for patterning, coating, and functional modification [8, 9]. Interestingly, natural marine organisms such as barnacles, mussels, and sandcastle worms have long inspired adhesives research—these organisms harness a number of highly diverse proteins to fulfill their needs for robust underwater adhesion [10-14].

Scientifically, the study of marine adhesive systems has led to major advances in our understanding about the interactions that facilitate their adhesion, which has driven the development of artificial underwater adhesives that exploit different adhesion principles inspired by nature including (i) like-charged adhesive polyelectrolytes based on cation-π interactions [15]; (ii) 3, 4-dihydroxyphenylalanine (Dopa)-based synthetic adhesive proteins and polymers integrating interfacial and cohesive interactions [16-20]; (iii) biomimetic positive- or negative-charge-induced adhesive coacervates building on electrostatic interactions [21, 22]; (iv) solid-liquid adhesive polymer mixes taking advantage of dipole-dipole interactions [23]. Additionally, inspired by the discovery of amyloid fibrous structures within barnacle cements [24], self-assembling multi-protein amyloid-like structures have been developed as robust adhesive coatings [25]. Despite these advances, the current state-of-the-art in this research area has not yet exploited the full potential of natural underwater adhesive systems, particularly their living and dynamic attributes [26]. For instance, mussel adhesion requires complex spatial-temporal regulation of highly diverse mussel foot proteins (mfps) mediated by cellular machineries [2, 27, 28]. Similarly, to function properly underwater, the multi-component adhesive proteins of both barnacles and sandcastle worms must undergo several successive controlled biological processes, including translation, secretion, delivery, and curing [29-31].

Here, we report the development of “living biofilm glues” that employ the dynamic biological processes that only live cells can undertake by leveraging tools from both genetic engineering and materials science. We rationally integrated the adhesion principles underlying the functional components of natural marine adhesive systems (FIG. 1a) to create functional cellular glues with strong adhesive performance, regenerative capacity, and environmental tolerance and we tested these biofilm glues, first individually and then in integrated functional cellular glues. We started with an engineered amyloid protein functionalized with a marine (e.g. mussel) adhesive protein to increase adhesion, and then added an engineered surface layer protein functionalized with a peptide known to induce coacervation and improve adhesion [32, 33]. We also demonstrate that adhesion performance of our living glues can be tuned through the inducible enzymatic modification of tyrosine-rich domains of the engineered proteins in the biofilms. Finally, we show that curing treatments with a variety of metal ions can further improve the adhesion capacity of our functional cellular glues (FIG. 1b).

### SUMMARY OF THE INVENTION

The invention provides engineered Bacillus subtilis biofilms as glues and related compositions and methods. Engineered living glues made by bacteria (e.g. Bacillus subtilis) biofilms integrate natural marine adhesive (e.g. mussel foot) proteins and electrostatic interactions into bacterial biofilm components such as amyloid protein TasA, surface layer protein BslA, and exopolysaccharides (EPS). The adhesion performances of living glues are tunable based on the components in the engineered biofilms. The living cellular glues can recapitulate the dynamic and living attributes of natural marine underwater adhesives, features that are missing in synthetic adhesives (e.g. polymeric adhesives or protein-based adhesives). These developed living glues exhibit evolvable, environmental tolerant, and self-regenerative properties. This is the first demonstration of living cellular glues. The living cellular glues are exemplified with Bacillus subtilis, a FDA approved GRAS (generally regarded as safe) strain, and the same design strategy of living glues can be applied to other bacterial systems including human probiotics (e.g. acetobacteria, lactobacillus, and saccharomycetes). Applications include wound dressing (living glues as bandages that can secret growth factors and therapeutic factors for wound healing), piipeline/underwater setting repairs (living glues with environmental responsiveness for corrosion detection, protection and inhibition under water settings), in vivo biomedical treatment (living glues that can secret therapeutic drugs and maintain the normal balance of gut flora), etc.

In an aspect the invention provides a fusion protein comprising a Bacillus biofilm-surface layer protein A (BslA) and a mussel foot protein 3S (Mfp3S) or coacervation inducing peptide thereof.

In embodiments: the BslA is (B. subtilis), Ba_BslA (B. amyloliquefaciens), Bl_BslA (B. licheniformis), or Bp_BslA (B. pumilus) and/or the peptide is a Mfp3S derived peptide such as Mfp3Sp, e.g. Wei et al. Adv Funct Mater. 2016 May 24; 26(20): 3496-3507.

In an aspect the invention provides glue composition comprising an engineered Bacillus biofilm comprising:

a) a first fusion protein comprising a Bacillus biofilm-surface layer protein A (BslA) and a mussel foot protein 3S (Mfp3S) or coacervation inducing peptide thereof;

b) a second fusion protein comprising an amyloid protein functionalized with a marine adhesion protein; and

c) exopolysaccharide (EPS).

In embodiments:

the Bacillus is Bacillus subtilis, Bacillus amyloliquefaciens, Bacillus licheniformis, or Bacillus pumilus; see, e.g. Morris et al., 2017, Scientific Reports 7, 6730;

the amyloid protein is TasA (B. subtilis), CsgA amyloid (E. coli), PSMs amyloid (S. aureus), or FapC (Pseudomonas spp.); see, e.g. Agustina Taglialegna, et al. 2016, J. Bacteriol, DOI: 10.1128/JB.00122-16;

the marine adhesion protein is muscle foot adhesive proteinMfp5, Mefp3, Mcfp3, or Mcfp5;

one or both of the fusion proteins comprise dihydroxyphenylalanine (DOPA) residues;

the composition is cured with one or more metal ions (e.g. Ca²⁺, Mg²⁺, Fe³⁺);

the composition comprises living, growing Bacillus microbes; and/or

the composition comprising viscoelasticity sufficient to be injectable into crevices or holes, and/or self-regenerative ability to maintain adhesive strength after multiple generations/passages.

In an aspect the invention provides a recombinant gene encoding a fusion protein comprising a Bacillus biofilm-surface layer protein A (BslA) and a mussel foot protein 3S (Mfp3S) or coacervation inducing peptide thereof.

In an aspect the invention provides a engineered Bacillus microbe comprising:

a) a first recombinant gene encoding a fusion protein comprising a Bacillus biofilm-surface layer protein A (BslA) and a mussel foot protein 3S (Mfp3S) or coacervation inducing peptide thereof; and

b) a second recombinant gene encoding a first fusion protein comprising an amyloid protein and a marine adhesion protein.

In embodiments: the microbe further comprising a recombinant, heterologous, inducible gene encoding a tyrosinase which catalyzes the hydroxylation of tyrosine residues to form Dopa on one or both of the fusion proteins.

In an aspect the invention provides a method of making a subject glue composition comprising growing a subject microbe under conditions wherein the biofilm is formed.

The invention includes all combinations of recited particular embodiments as if each combination had been laboriously recited.

### BRIEF DESCRIPTION OF THE FIGURES

FIG. 1a. Marine adhesive systems that employ distinct adhesion principles. Left, barnacles using cement proteins containing amyloid-like structures. Middle, mussels using adhesive foot proteins possessing Dopa, tyrosine, and lysine residues that contribute to interfacial adhesion, cation-π mediated self-coacervation and cohesion. Right, sandcastle worms using oppositely charged polyelectrolytes-induced complex coacervates.

FIG. 1b. Design for functional cellular glues based on engineered B. subtilis biofilms containing adhesive components inspired by three natural marine systems. Top, Conceptual illustration of an integrated biofilm-based functional cellular glue on a substrate, showing bacterial cells embedded inside a functionalized extracellular matrix that is rich in engineered amyloid structural proteins fused with a mussel foot protein (TasA-Mefp5), engineered biofilm surface proteins fused with an engineered mussel-derived peptide (BslA-Mfp3Sp), exopolysaccharides (EPS), and metal ions. Bottom left, a TasA-Mefp5 fusion adhesion system. Bottom middle, BslA-Mfp3Sp fusion adhesion system. Bottom right, adhesion based on electrostatic interactions occurring among EPS, metal ions, and the modified DOPA-rich adhesive domains of the TasA-Mefp5 fusion protein.

FIG. 2a. Functional cellular glues with engineered amyloid nanofibre network and biofilm surface protein: The bslA⁻ tasA⁻ eps⁻ B. subtilis mutant strain carrying a plasmid harboring tasA-mefp5.

FIG. 2b. TEM images of TasA-immuno-gold labeled biofilms produced by the bslA⁻ tasA⁻ eps⁻ (left), bslA⁻ tasA⁻ eps⁻/TasA (middle) and bslA⁻ tasA⁻ eps⁻/TasA-Mefp5 strains (right). The rectangles representing the areas are shown at higher magnification at the bottom, respectively.

FIG. 2c. Storage modulus of the bslA⁻ tasA⁻ eps⁻, bslA⁻ tasA⁻ eps⁻/TasA, and bslA⁻ tasA⁻ eps⁻/TasA-Mefp5 biofilms as a function of strain amplitude at constant frequency (ω=10 rad/s).

FIG. 2d. Lap shear adhesion measurement for the bslA⁻ tasA⁻ eps⁻, bslA⁻ tasA⁻ eps⁻/TasA, and bslA⁻ tasA⁻ eps⁻/TasA-Mefp5 biofilms.

FIG. 2e. The B. subtilis bslA⁻ strain carrying a plasmid harboring bslA-mfp3Sp.

FIG. 2f. Light and fluorescence microscopy of the biofilm (produced by the bslA⁻ strain containing a plasmid harboring bslA-mfp3Sp (left) and bslA-mfp3Sp-spytag (right)) incubated with purified GFP-Spycatcher protein.

FIG. 2g. Storage modulus of the bslA⁻, bslA⁻/BslA, and bslA⁻/BslA-Mfp3Sp biofilms as a function of strain amplitude at constant frequency (ω=10 rad/s). The dotted lines indicating the boundary of the ‘linear viscoelastic region’ for a given biofilm using 5% change of the initial value of storage modulus as the threshold.

FIG. 2h. Lap shear adhesion measurement for the bslA⁻, bslA⁻/BslA, and bslA⁻/BslA-Mfp3Sp biofilms. All lap shear results are means±s.e.m. of five replicate samples from biologically independent biofilm cultures.

FIG. 3a. Integrated, modified, and cured cellular glues: Schematic showing the step-by-step (sequential) construction of cellular glues, via 1) integration of the tasA-mefp5 and bslA-mfp3Sp genes into the B. subtilis (“Tyr-BS”) genome, resulting in functional biofilms with adhesive properties derived from Mefp5 (the protein) and the Mfp3Sp peptide to make “Tyr-BS cellular glues”; 2) Transformation of an IPTG-inducible plasmid harboring a tyrosinase gene into Tyr-BS cells to make modified “Dopa-BS cellular glues”; expression of this tyrosinase results in the in vivo modification of tyrosine residues in the adhesive domains into Dopa residues in the biofilms; 3) Enzyme-modified functional biofilms treated with external metals ions (Mg²⁺, Ca²⁺, or Fe³⁺) to cure the functional cellular glues to make “Metal ions-cured Dopa-BS cellular glues”.

FIG. 3b. Photograph and SEM images of Tyr-BS, Dopa-BS, and Fe³⁺-cured Dopa-BS cellular glues (from top to bottom: Tyr-BS cellular glue (i and ii), Dopa-BS cellular glue (iii and iv), and Fe³⁺-cured Dopa-BS cellular glue (v and vi).

FIG. 3c. Storage modulus of a series of functional cellular glues as a function of strain amplitude at constant frequency (ω=10 rad/s). The dotted lines indicating the boundary of the ‘linear viscoelastic region’ for a given biofilm using 5% change of the initial value of storage modulus as the threshold.

FIG. 3d. Adhesive strength comparison of the wild-type biofilms and the Tyr-BS, Dopa-BS, and Fe³⁺-cured Dopa-BS cellular glues measured in lap shear tests.

FIG. 4a. Lap shear adhesive capacity of biofilm cellular glues assessed under different conditions: Shear adhesive strengths of the wild type (WT), Tyr-BS, Dopa-BS and Fe³⁺-cured Dopa-BS biofilm glues on different substrates (PTFE, aluminum foil, and PET).

FIG. 4b. Shear adhesive strength of the ion-solution-cured Dopa-BS biofilms assessed by curing with 2 μL CaCl₂, MgCl₂, or FeCl₃ solution of varied ion concentrations.

FIG. 4c. Shear adhesive strength of the Dopa-BS and Fe³⁺-cured Dopa-BS biofilms assessed at different shear speeds.

FIG. 4d. Shear adhesive strength of the metal ion-cured Dopa-BS biofilms assessed after exposure to solutions of different pH values (HCl (pH 1.0), NaCl (7.0), and NaOH (pH 12.0)).

FIG. 5a. Functional performance, self-regeneration, and practical applications of functional cellular glues: Adhesion strength comparison between functional cellular glues and wild-type biofilms tested under different humidity levels or after exposure to different detergents (0.1% m/v SDS or urea 8 M urea solution).

FIG. 5b. Adhesive strength of functional cellular glues (wild-type and Dopa-BS cellular glues) for samples of five successive generations (passages); note: inserted graphs represent contact angle assay results for corresponding Dopa-BS cellular glues.

FIG. 5c. Application of functional cellular glues to fill and repair crevices on a PDMS substrate: from left to right, (i) damaged crevices on PDMS, (ii) filling the crevices with the glues, (iii) curing the filled glues with solution containing Fe³⁺ ions, (iv) submersion in water.

FIG. 5d. Environmental tolerance of the repaired PDMS crevices after exposure to harsh conditions: (left) organic solvent (methanol: CH₃OH), (middle) HCl solution (pH=3.0), (right) NaOH solution (pH=12.0).

FIG. 6a. AFM Phase images of the bslA⁻ tasA⁻ eps⁻ (a), bslA⁻ tasA⁻ eps⁻/TasA.

FIG. 6b. bslA⁻ tasA⁻ eps⁻/TasA-Mefp5. The white arrows indicate the presence of extracellular TasA and TasA-Mefp5 nanofibres surrounding the cells.

FIG. 6c. biofilms with surface features. The white arrows indicate the presence of extracellular TasA and TasA-Mefp5 nanofibres surrounding the cells.

FIG. 7. High resolution TEM (TEM) and corresponding EDS analysis of the TasA-immuno gold labelling nanoparticles within the bslA⁻ tasA⁻ eps⁻/TasA biofilm. Gold nanoparticles bound to TasA protein-containing nanofibres (i) were further illustrated in details (ii and iii). EDS mapping clearly revealed the nanoparticles contain the Au element, as indicated by the clearly matched area between the morphological and element mapping images (iv-vii).

FIG. 8. High resolution TEM (TEM) and corresponding EDS analysis of the TasA-immuno gold labelling nanoparticles within the bslA⁻ tasA⁻ eps⁻/TasA-Mefp5 biofilm. Gold nanoparticles bound to TasA-Mefp5 protein-containing nanofibres (i) were further illustrated in details (ii and iii). EDS mapping clearly revealed the nanoparticles contain the Au element, as indicated by the clearly matched area between the morphological and element mapping images (iv-vii).

FIG. 9a. TEM images of the two differently functionalized biofilms labeled with NTA-decorated gold nanoparticles: (a) bslA⁻ tasA⁻ eps⁻/TasA biofilm containing TasA proteins lacking His-tag.

FIG. 9b. bslA⁻ tasA⁻ eps⁻/TasA-Mefp5 biofilm containing C-terminal His-tagged TasA-Mefp5 protein.

FIG. 10. Congo Red (CR) absorbance with bslA⁻ tasA⁻ eps⁻, bslA⁻ tasA⁻ eps⁻/TasA and bslA⁻ tasA⁻ eps⁻/TasA-Mefp5 biofilm pellets Enhanced absorption of Congo Red was observed in the bslA⁻ tasA⁻ eps⁻/TasA and bslA⁻ tasA⁻ eps⁻/TasA-Mefp5 biofilm pellets compared to bslA⁻ tasA⁻ eps⁻ control strain, confirming the successful production of TasA and TasA-Mefp5 amyloid proteins in the bslA⁻ tasA⁻ eps⁻/TasA and bslA⁻ tasA⁻ eps⁻/TasA-Mefp5 biofilm samples.

FIG. 11a. Viscoelastic properties of the bslA⁻ tasA⁻ eps⁻ biofilm: G′ (storage modulus) and G″ (loss modulus) as a function of strain amplitude at constant frequency (ω=10 rad/s).

FIG. 11b. Shear stress (a).

FIG. 11c. Biscosity (i) as a function of applied shear rate.

FIG. 12a. Viscoelastic properties of the bslA⁻ tasA⁻ eps⁻/TasA biofilm: G′ (storage modulus) and G″ (loss modulus) as a function of strain amplitude at constant frequency (ω=10 rad/s).

FIG. 12b. Shear stress (a) as a function of applied shear rate.

FIG. 12c. Viscosity (i) as a function of applied shear rate.

FIG. 13a. Viscoelastic properties of the bslA⁻ tasA⁻ eps⁻/TasA-Mefp5 biofilm: G′ (storage modulus) and G″ (loss modulus) as a function of strain amplitude at constant frequency (ω=10 rad/s).

FIG. 13b. Shear stress (a).

FIG. 13c. Viscosity (i) as a function of applied shear rate.

FIG. 14a. Lap shear measurements and adhesion strength of engineered biofilm glue: A schematic illustration of the lap shear test.

FIG. 14b. Lap shear measurement setup based on an Instron 5966 instrument.

FIG. 14c. Typical force-distance curves of the bslA⁻ tasA⁻ eps⁻/TasA-Mefp5 biofilm glue, and the inserted showing the typical method and corresponding equation for determination of the adhesive strength based on the force-distance curves.

FIG. 15a. Plasmid construction of pHT-tyrosinase-tasA-mefp5 plasmid.

FIG. 15b. Shear adhesive strength comparison for the bslA⁻ tasA⁻ eps⁻/TasA-Mefp5 and bslA⁻ tasA⁻ eps⁻/Tyrosinase-TasA-Mefp5 biofilms.

FIG. 16a. Viscoelastic properties of the bslA⁻ tasA⁻ eps⁻/Tyrosinase-TasA-Mefp5 biofilm: G′ (storage modulus) and G″ (loss modulus) as a function of strain amplitude at constant frequency (ω=10 rad/s).

FIG. 16b. Shear stress (a).

FIG. 16c. Viscosity (i) as a function of applied shear rate.

FIG. 17. Light and fluorescence microscopy of the biofilm (produced by the bslA⁻ strain containing a plasmid harboring bslA-mfp3Sp (left) and bslA-mfp3Sp-spytag (right)) incubated with purified mCherry-Spycatcher protein.

FIG. 18. Macro-morphology and water contact angle assay of the bslA⁻, bslA⁻/BslA, and bslA⁻/BslA-Mfp3Sp biofilms.

FIG. 19a. Viscoelastic properties of the bslA⁻ biofilm: G′ (storage modulus) and G″ (loss modulus) as a function of strain amplitude at constant frequency (ω=10 rad/s).

FIG. 19b Shear stress (a).

FIG. 19c. Viscosity (i) as a function of applied shear rate.

FIG. 20a. Viscoelastic properties of the bslA⁻/BslA biofilm: G′ (storage modulus) and G″ (loss modulus) as a function of strain amplitude at constant frequency (ω=10 rad/s).

FIG. 20b. Shear stress (a).

FIG. 20c. Viscosity (i) as a function of applied shear rate.

FIG. 21a. Viscoelastic properties of the bslA⁻/BslA-Mfp3Sp biofilm: G (storage modulus) and G′ (loss modulus) as a function of strain amplitude at constant frequency (ω=10 rad/s).

FIG. 21b. Shear stress (a).

FIG. 21c. Viscosity (i) as a function of applied shear rate.

FIG. 22. The shear adhesive strength comparison of the bslA⁻ tasA⁻ eps⁻, bslA⁻ tasA⁻ and Fe³⁺-cured bslA⁻ tasA⁻ biofilms based on lap shear measurement. The EPS-containing bslA⁻ tasA⁻ biofilm exhibited larger adhesive strength compared to the EPS-lacking bslA⁻ tasA⁻ eps⁻ biofilm.

FIG. 23. SEM and corresponding EDS of the bslA⁻ tasA⁻ biofilm sample added with or without FeCl₃ solution. Compared to the bslA⁻ tasA⁻ biofilm alone (i), the addition of FeCl₃ solution caused a drastic condensation of biofilm matrix (ii) in SEM images. Further EDS analysis illustrated that the condensed area of biofilm matrix was rich in O, Fe, C, and P elements (iii-vi).

FIG. 24. FTIR spectra of the bslA⁻ tasA⁻ eps⁻, bslA⁻ tasA⁻ biofilms in the presence or absence of FeCl₃ solution. Peaks of remarkable differences were highlighted and annotated with specific reference peaks [8]. The peaks at 1635 cm⁻¹ (—NH₂), 1542 cm⁻¹ (—CO—NH—), and 1400 cm⁻¹ (—OH) displayed a strong and broadened absorbance in all four biofilm samples. The peaks at 1598 cm⁻¹ (—COO⁻), 1117 cm⁻¹ (—P═O), 1264 cm⁻¹ (—PO₃²), 1084 cm⁻¹ (—PO₃²), and 979 cm⁻¹ (PO₃²⁻) displayed an intensive absorbance only in the EPS-containing bslA⁻ tasA⁻ biofilm. The peaks at 1170 cm⁻¹ (—C—O—C—) and 1042 cm⁻¹ (—P—OFe) displayed a broadened and intense absorbance in the FeCl₃ containing bslA⁻ tasA⁻ biofilm. vₐₛ: asymmetric stretching vibration; vₛ: symmetric stretching vibration.

FIG. 25a. Confirmation of tyrosinase expression in Dopa-BS biofilm and influence of enzymatic modification by tyrosinase on the adhesion strength of engineered Dopa-BS biofilm glues: NBT assay comparison between the uninduced Dopa-BS biofilm and Dopa-BS biofilm expressing tyrosinase (following IPTG induction) confirmed the detection of Dopa residues in the Dopa-BS biofilms that expressed tyrosinase. Western blotting also showed that the Dopa-BS biofilm sample contain expressed tyrosinase upon IPTG induction.

FIG. 25b. The adhesive strength of Dopa-BS biofilm (upon IPTG induction) was significantly improved compared to the Dopa-BS biofilm (without IPTG), supporting the hypothesis that enzymatic modification of Dopa-BS biofilm matrix contributed to the improved adhesion strength. (Note: the B. subtilis strain 2569::tasA-mefp5::bslA-mfp3Sp/pHT-tyrosinase is denoted as modified B. subtilis “Dopa-BS”).

FIG. 26. Digital photographs showing typical morphologies of the Tyr-BS and Dopa-BS (induced by IPTG) biofilms.

FIG. 27a. Viscoelastic properties of the Tyr-BS biofilm: G′ (storage modulus) and G″ (loss modulus) as a function of strain amplitude at constant frequency (ω=10 rad/s).

FIG. 27b. Shear stress (a).

FIG. 27c. Viscosity (i) as a function of applied shear rate.

FIG. 28a. Viscoelastic properties of the Dopa-BS biofilm: G′ (storage modulus) and G″ (loss modulus) as a function of strain amplitude at constant frequency (ω=10 rad/s).

FIG. 28b. Shear stress (a).

FIG. 28c. Viscosity (i) as a function of applied shear rate.

FIG. 29a. Viscoelastic properties of the Fe³⁺-cured Dopa-BS biofilm: G′ (storage modulus) and G″ (loss modulus) as a function of strain amplitude at constant frequency (ω=10 rad/s).

FIG. 29b. Shear stress (a).

FIG. 29c. Viscosity (i) as a function of applied shear rate.

FIG. 30a. Illustration of the 90° peel test.

FIG. 30b. Corresponding data (force F, normalized by the tape width b, as a function of peel length) of the Dopa-BS biofilms on different substrates including: aluminum (Al) plate, polyethylene terephthalate (PET) plastic, and glass substrates.

FIG. 31. Self-regeneration of Dopa-BS biofilm glues. Digital graphs showing identical morphologies of the self-regenerated biofilm glues by scraping the cultures and passaging them through five successive cycles of re-inoculation and re-growth on agar plates containing MSgg culture media (from left to right).

FIG. 32. Self-regeneration of the wild-type biofilm glues. Digital photographs and corresponding contact angle assay results showing identical morphologies and hydrophobicity of the self-regenerated biofilm glues by scraping the cultures and passaging them through five successive cycles of re-inoculation and re-growth on agar plates containing MSgg culture media (from left to right).

FIG. 33a. Application demonstration of engineered biofilms glues in wet conditions: A glass slide adhered onto a thread with the use of Fe³⁺-cured Dopa-BS biofilm, could resist falling down in aqueous solution.

FIG. 33b. A glass slide adhered to an aluminum plate with the use of Fe³⁺-cured Dopa-BS biofilm (2×2 cm overlapped area), could hang a 50 g weight without falling down in aqueous solution.

FIG. 34a. The injectability of engineered biofilm glues: Dopa-BS biofilm glues stored in a syringe.

FIG. 34b. The letter “B. subtilis” by manual injection of the Dopa-BS biofilm glues directly onto a glass substrate (right).

FIG. 35. The shear adhesive strength comparison of the Fe³⁺-cured Dopa-BS biofilms after treatment with HCl, NaOH, or CH₃OH solutions. Note: the control is the original Fe³⁺-cured Dopa-BS biofilm without any treatment.

FIG. 36. Plasmid maps of the pHT01 plasmid, the constructed pHT-tapA-sipW-tasA and pHT-tapA-sipW-tasA-mefp5 plasmids.

FIG. 37. Plasmid maps of the constructed pHT-bslA, pHT-bslA-mfp3Sp, and pHT-bslA-mfp3Sp-spytag plasmids.

FIG. 38. Plasmid maps of the constructed pHT-tyrosinase and pET22b-GFP-spycatcher plasmids.

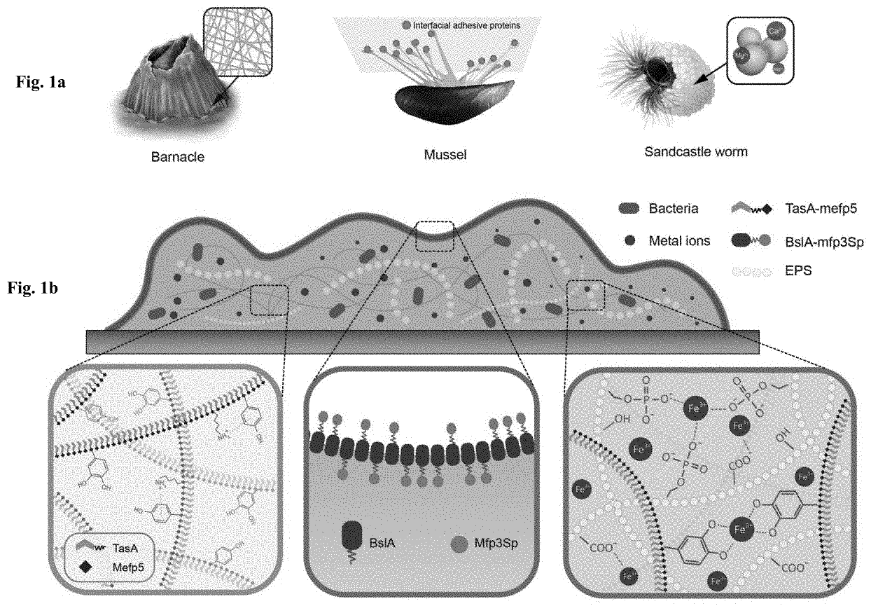

### DESCRIPTION OF PARTICULAR EMBODIMENTS OF THE INVENTION

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

The powerful biological adhesion systems of barnacles, mussels, and other marine animals have led to bio-inspired adhesives with impressive performance characteristics. However, lacking the capacities that only cells possess (e.g., self-regeneration, environmental responsiveness, etc.), these artificial materials cannot exploit the full potential of the natural adhesive systems that inspired them. We have developed and disclose here “functional cellular glues” made of microbial (e.g Bacillus subtilis) biofilms—tightly associated bacteria encased in an extracellular matrix—that contain both an engineered amyloid protein functionalized with a marine bioadhesive (e.g. mussel foot) protein and an engineered hydrophobin-like protein. We demonstrate proof-of-concept for both tunable adhesion performance via inducible enzymatic modification and for improved adhesion through metal ion-assisted curing. By conceptualizing biofilms themselves—rather than individual material components as adhesives, we have developed a malleable engineering platform enabling smart living glues with dynamic, self-healing, and other previously unattainable material properties.

Results and Discussion

We selected B. subtilis biofilms as our initial engineering platform, because unlike E. coli or other Gram-negative bacteria, B. subtilis has only one outer membrane, a feature that has long made this bacterium popular for the production of secreted enzymes and other large proteins (even at the industrial scale [34, 35]). Among other components, B. subtilis biofilms contain TasA amyloid fibres [36], the hydrophobic surface layer protein BslA [37], and structurally complex exopolysaccharide (EPS) that are the ultimate biosynthetic products of the epsA-O operon (hereafter referred to eps genes) [38]. We designed and tested glues based on each of these genes, starting with TasA. TasA is an amyloidogenic protein that is amenable to genetic elaboration. In particular, this family of proteins can accommodate heterologous peptide or protein domain inserts, thereby enabling new functionalities while maintaining the ability to self-assemble into nanofibres [39, 40]; further, previous in vitro study had shown that stronger underwater adhesion could be achieved with nanofibres comprising CsgA-Mfp5 fusion proteins than with CsgA nanofibres only (major protein components of E. coli biofilms) [25]. We therefore designed a fusion protein consisting of TasA, a peptide linker, and the mussel foot protein Mefp5.

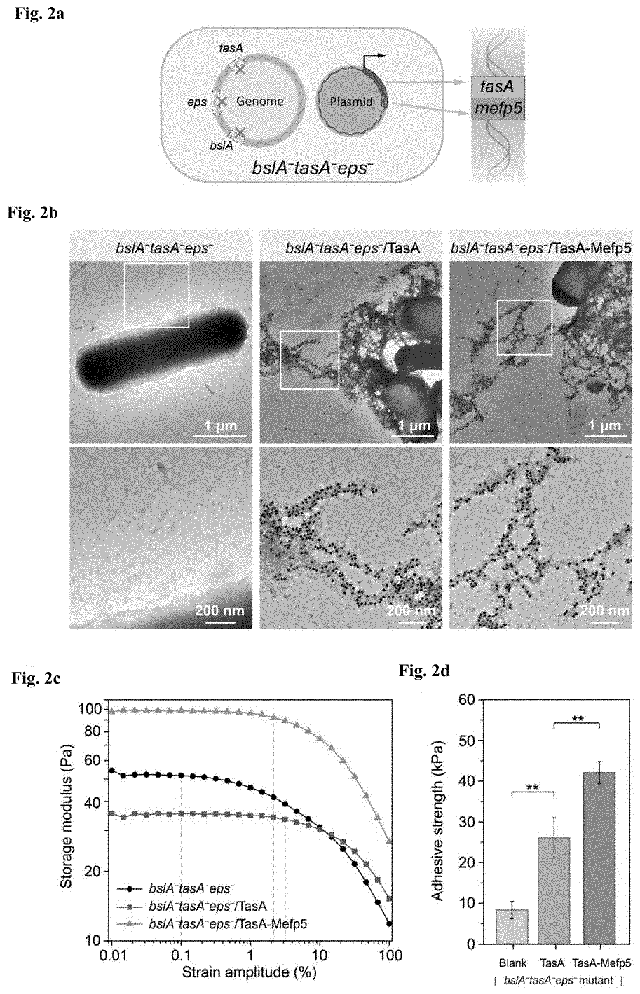

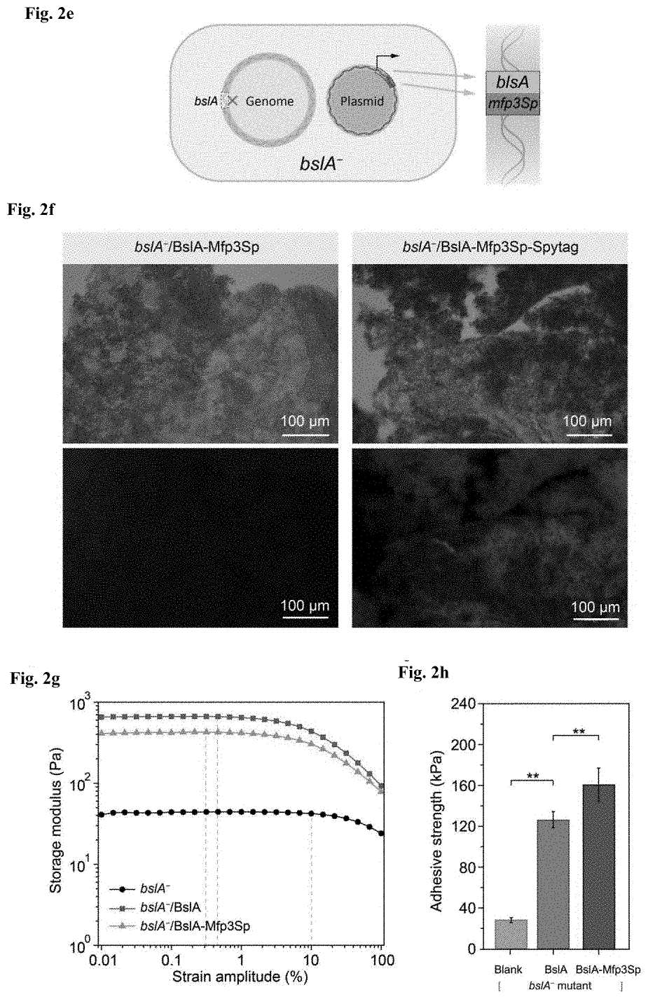

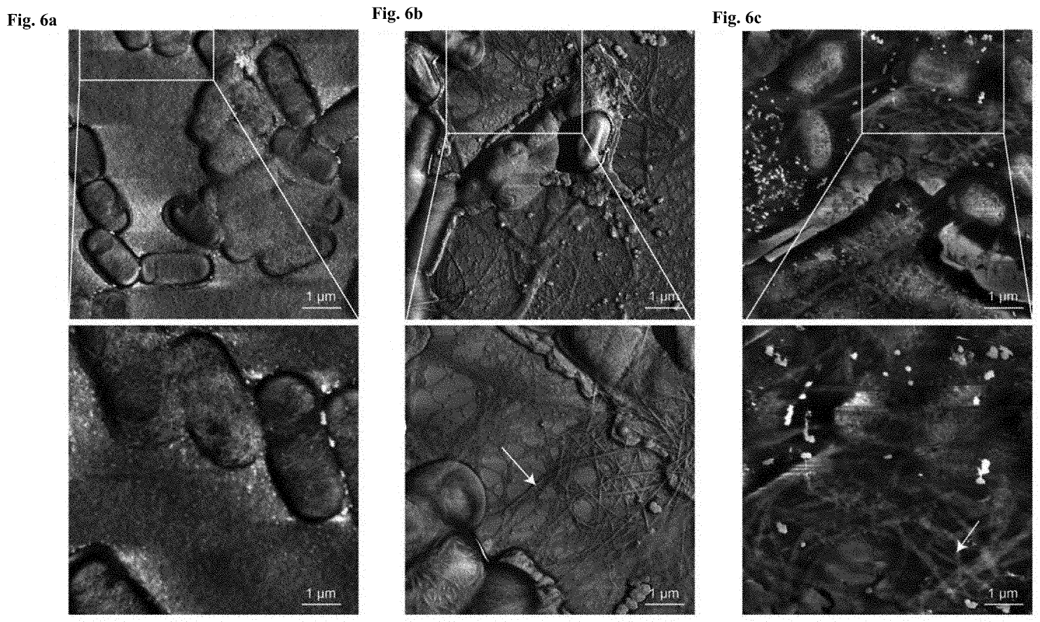

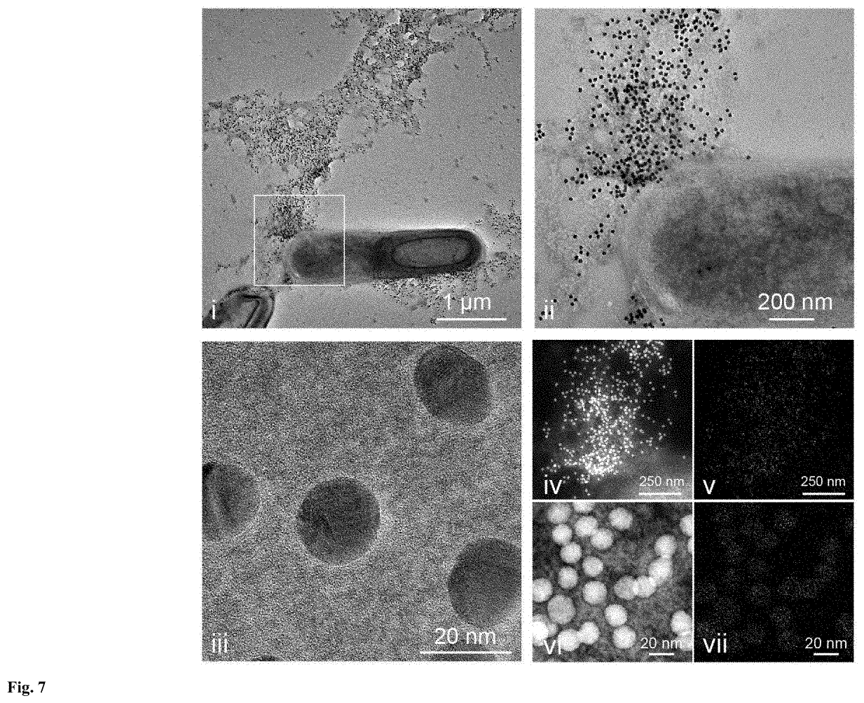

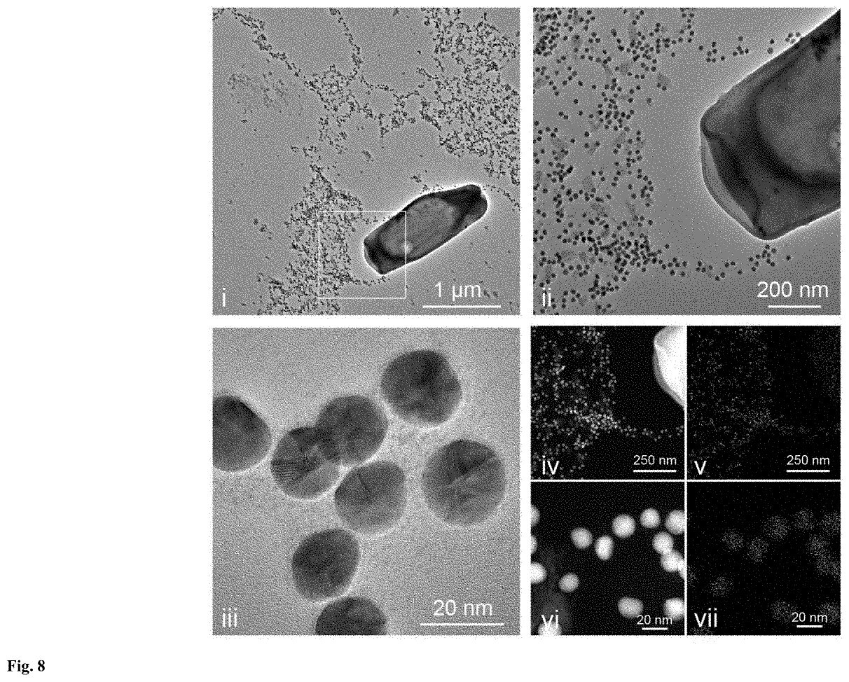

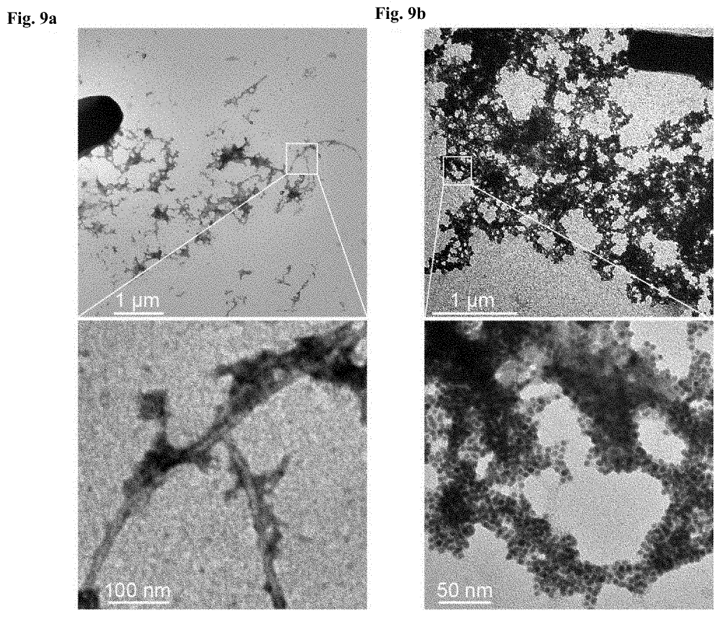

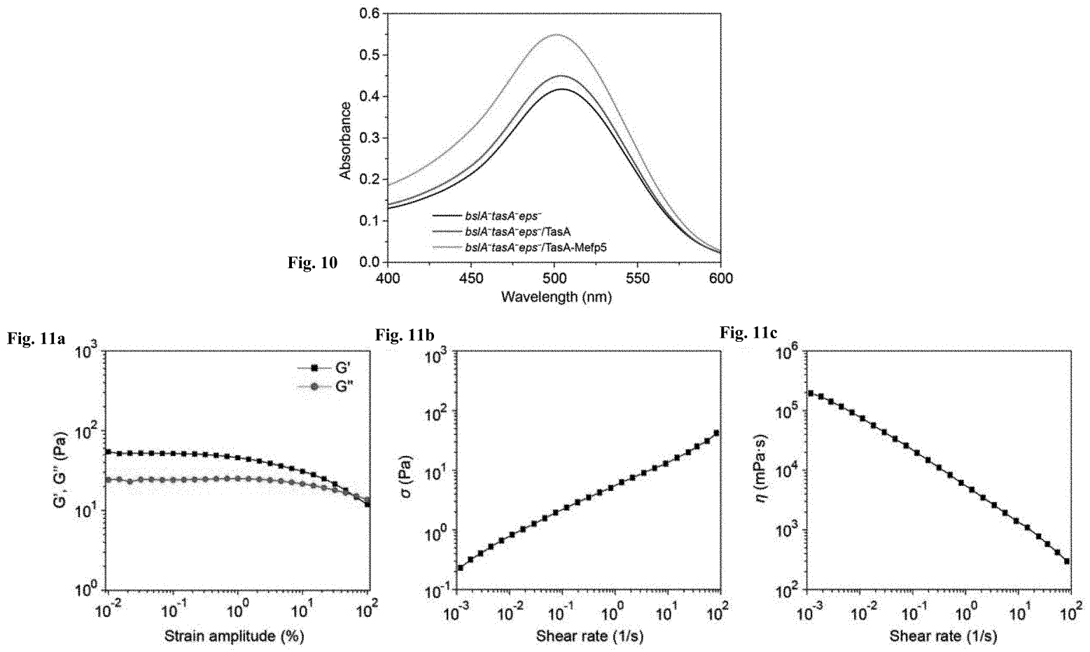

To test the TasA-Mefp5 fusion protein, we initially generated a B. subtilis strain that lacked the tasA, bslA, and eps genes (denoted as bslA⁻ tasA⁻ eps⁻) (FIG. 2a), and confirmed that the bslA⁻ tasA⁻ eps⁻ mutant strain did not produce normal biofilms. We subsequently transformed the bslA⁻ tasA⁻ eps⁻ mutant strain with plasmids containing gene fragments encoding TasA and TasA-Mefp5. Transmission electron microscopy (TEM) and atomic force microscopy (AFM) revealed the bslA⁻ tasA⁻ eps⁻ strain was able to secrete TasA and TasA-Mefp5 proteins that could self-assemble into extracellular nanofibres (FIGS. 2b and 6). Note that high resolution TEM (HRTEM) and corresponding energy dispersive X-ray spectroscopy (EDS) analyses together confirmed that immuno-gold labeled anti-TasA antibodies could specifically bind to the TasA-containing nanofibres (FIGS. 7 and 8). Additionally, nitrilotriacetic acid (NTA)-decorated gold nanoparticles also showed high affinity to the nanofibres composed of the C-terminal His-tagged TasA-Mefp5 proteins (FIG. 9). Taken together, these results implied that the full-length TasA-Mefp5 fusion proteins were expressed and assembled in the bslA⁻ tasA⁻ eps⁻/TasA-Mefp5 biofilm. These observations were in agreement with the results of Congo red assay, which revealed amyloid (TasA or TasA-Mefp5)-containing biofilms, compared to the deficient bslA⁻ tasA⁻ eps⁻ biofilm, indeed showed enhanced absorbance at the maximum characteristic peak of 502 nm (FIG. 10).

As bacterial biofilms typically exhibit gel-like structures [41], we next used rheological measurements to investigate the viscoelastic properties of engineered biofilms produced by the bslA⁻ tasA⁻ eps⁻, bslA⁻ tasA⁻ eps⁻/TasA, and bslA⁻ tasA⁻ eps⁻/TasA-Mefp5 strains. Specifically, we measured the storage modulus—representing the stiffness of a material—as a function of strain amplitude, which reveals the deformation of a material body. The range of strain amplitudes over which the storage modulus remains relatively constant is defined as the linear viscoelastic region, which represents the largest recoverable deformation that can be achieved without disrupting the material's structure (this can be conceptualized as the ‘resilience’ of a material [42]). Compared to the bslA⁻ tasA⁻ eps⁻ biofilm, the bslA⁻ tasA⁻ eps⁻/TasA biofilm had improved resilience but decreased stiffness. The bslA⁻ tasA⁻ eps⁻/TasA-Mefp5 biofilm exhibited higher stiffness yet similar resilience compared to the bslA⁻ tasA⁻ eps⁻/TasA biofilm (FIG. 2c and S6-8). We infer that the expression of TasA amyloid in the bslA⁻ tasA⁻ eps⁻ mutant strain helps to generate a biofilm scaffold which can relatively more easily become deformed but that also has a larger deformation amplitude under shear stress [43]; further, the adhesive Mefp5 fusion protein could be expected to result in more compact and dense biofilm matrix, potentially via cation-π and 7E-7E interactions [44, 45].

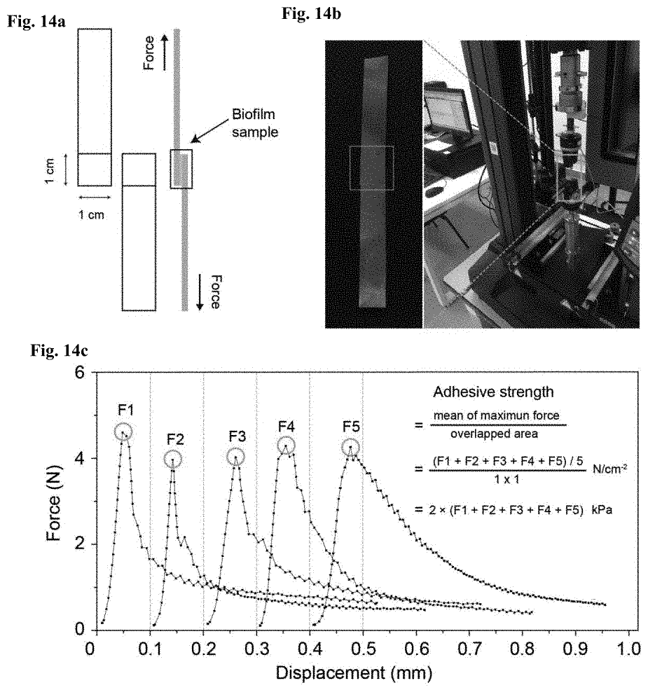

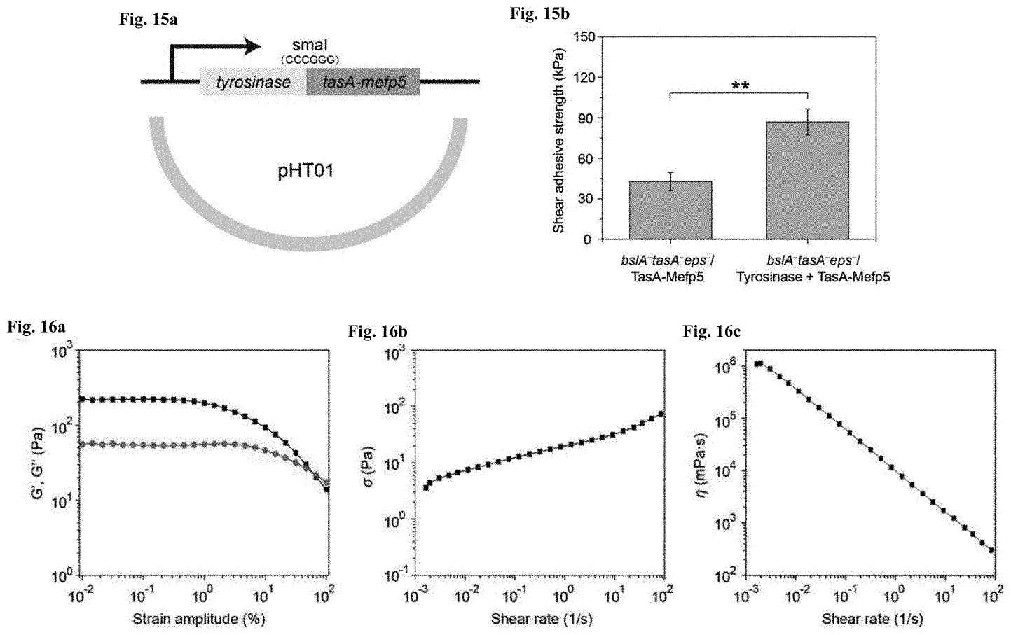

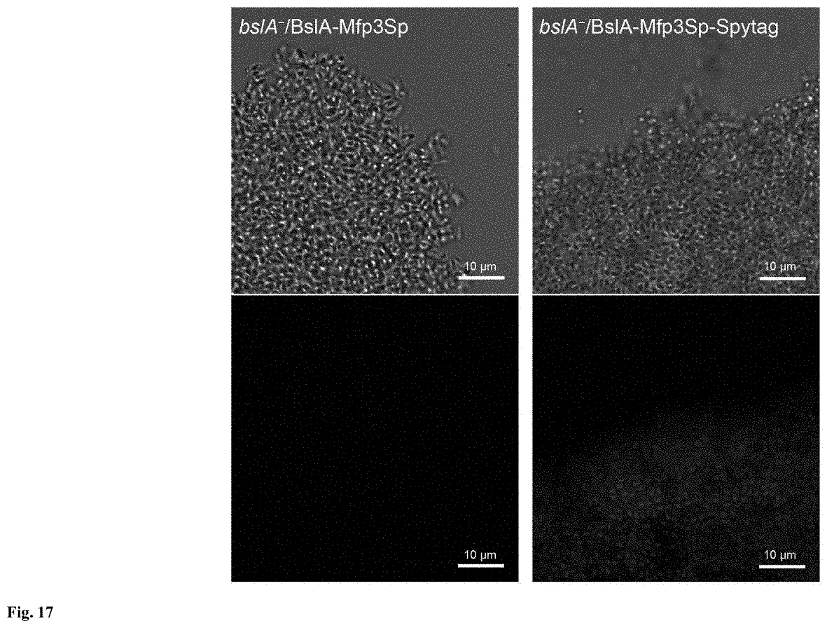

We measured the shear adhesive strength of the biofilms using lap shear tests and found that the bslA⁻ tasA⁻ eps⁻/TasA-Mefp5 biofilm exhibited the greatest shear adhesive strength (42.71±6.68 kPa), which was nearly five times stronger than the bslA⁻ tasA⁻ eps⁻ biofilm, and was 60% stronger than the bslA⁻ tasA⁻ eps⁻/TasA biofilm (FIGS. 2d and 14). Collectively, these results demonstrate that the TasA-Mefp5 fusion monomers can be secreted and can self-assemble into nanofibres and that the presence of the Mefp5 domain on these nanofibres improves both the storage modulus and the shear adhesive strength of biofilms. These results are consistent with the demonstrated ability of cation-π interactions within tyrosine-rich domains (e.g. Mefp5) that increase the adhesive capacity of materials [15, 44]. Furthermore, the shear adhesive strength and storage modulus of the TasA-Mefp5-containing biofilm can be further improved by additionally expressing a tyrosinase that can catalyze tyrosine to Dopa, such as through Dopa-enhanced interfacial (e.g. Dopa coordination to iron sheets) and cohesive interactions (e.g. covalent crosslinking) (FIGS. 15-16) [10].

Having tested the fusion protein-based functionalized amyloid nanofibres component of our functional cellular glue concept, and with the goal of further improving adhesive strength, we next generated a B. subtilis strain lacking the gene encoding the biofilm hydrophobin surface protein BslA (denoted as bslA⁻). This bslA⁻ mutant strain, used to test a fusion protein that we designed, contained BslA, a linker peptide, and Mfp3Sp. Mfp3Sp is a previously described tyrosine-rich adhesive peptide derived from the mussel foot protein Mfp3S that forms coacervate structures with low surface energy and thereby facilitates spreading over surfaces [46] (FIG. 2e). Our rationale underlying this fusion protein design is that the presence of the coacervate-promoting Mfp3Sp peptide on the B. subtilis biofilm surface layer protein BslA should in theory enhance biofilm interfacial adhesion and the BslA domain would prevent water penetration into biofilms [47], especially in wetting conditions.

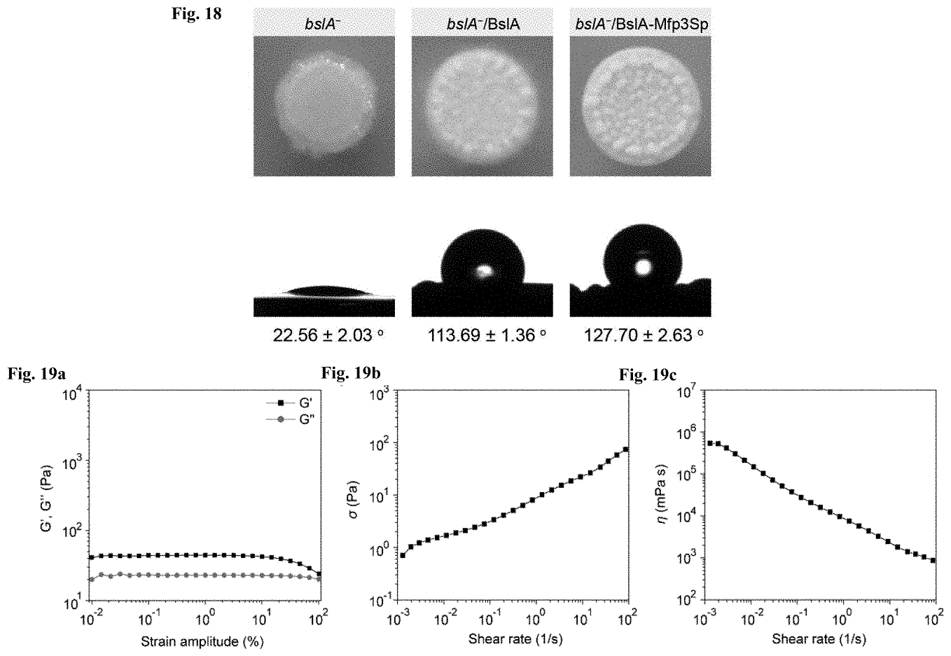

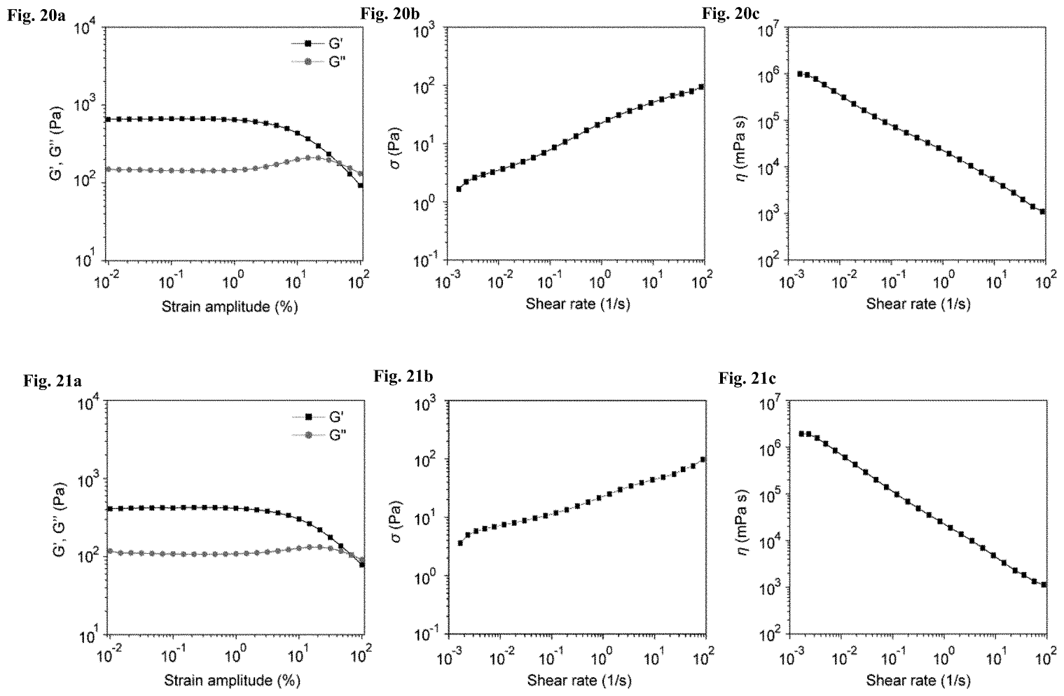

Prior to examining the effects of the BslA-Mfp3Sp fusion protein on adhesion, we initially generated a construct for a BslA-Mfp3Sp-Spytag fusion protein (to enable fluorescence labeling via a Spytag-Spycatcher protein-protein interaction [48]) and introduced it into the bslA⁻ strain to confirm that a functionalized BslA-Mfp3Sp fusion protein can be secreted into B. subtilis biofilms (FIGS. 2f and 17). Water contact angle assays revealed that the bslA⁻ strain formed a strong wrinkled and hydrophobic surface with the expression of BslA or BslA-Mfp3Sp proteins (FIG. 18). Rheological measurements indicated that the expression of BslA strongly enhanced the storage modulus of the bslA⁻ biofilm, potentially by improving biofilm surface hydrophobicity, roughness, and stiffness (FIGS. 2g, 19-20) [49]. Further, while the presence of the Mfp3Sp peptide did not strongly affect the stiffness and resilience of the engineered biofilms (FIGS. 2g and 21), this Mfp3Sp peptide did significantly improve the shear adhesive strength in the bslA⁻/BslA-Mfp3Sp biofilm over the bslA⁻/BslA biofilm (FIG. 2h). The shear adhesive strength values measured for both bslA⁻/BslA-Mfp3Sp and bslA⁻ BslA biofilms (˜160 and 127 kPa) were much greater than those for any of the biofilms produced by the bslA⁻ tasA⁻ eps⁻ strains (FIG. 2d, h). Thus, in addition to serving as the proof-of-concept demonstration for the use of the Mfp3Sp peptide in an in vivo system, these results confirm that this peptide can confer improved adhesion to substrates, by promoting biofilm interfacial adhesion through cation-π interactions within or between the tyrosine-rich Mfp3Sp domains [10, 15, 44].

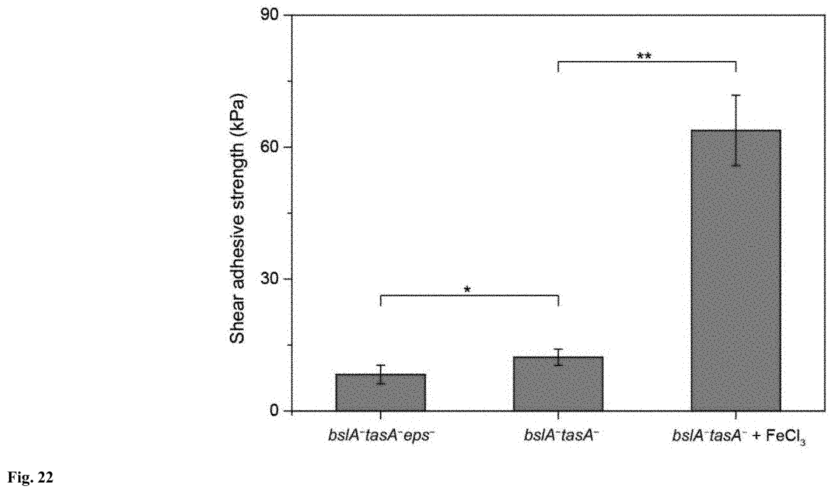

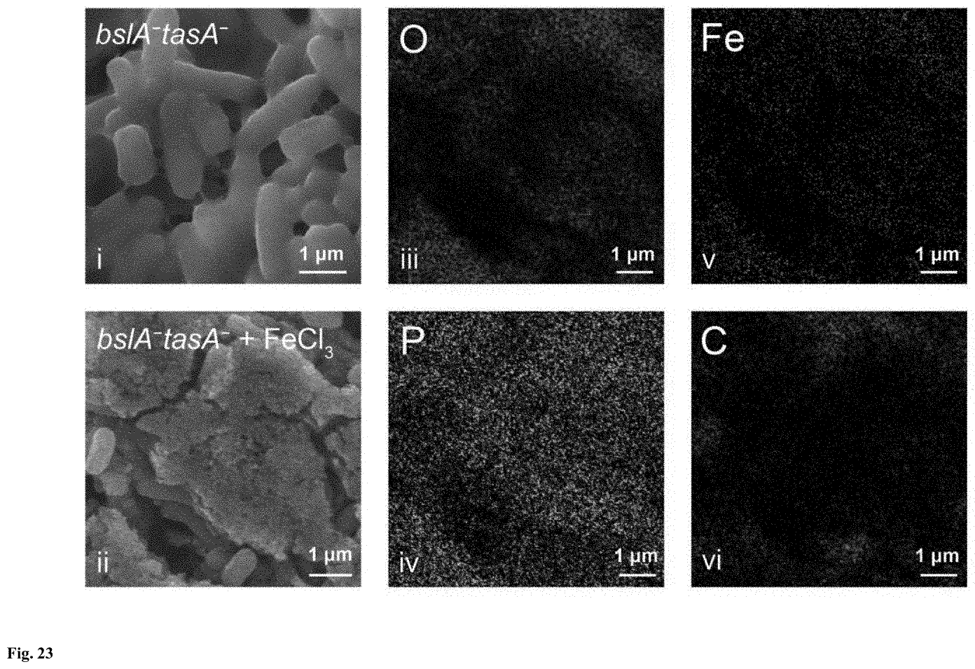

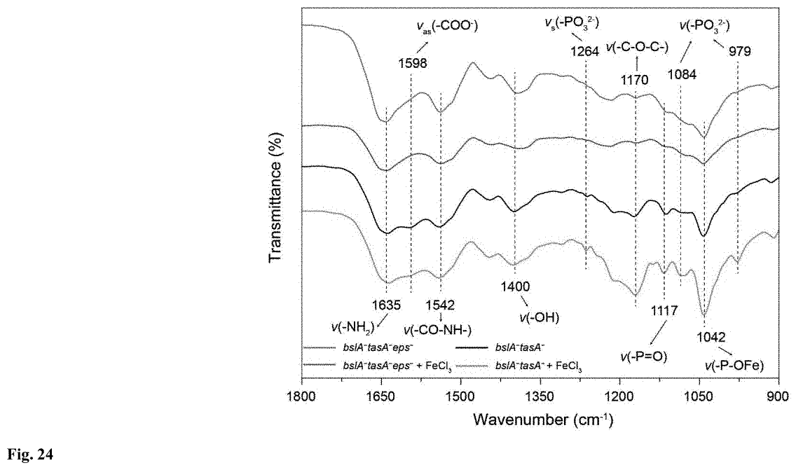

As noted above, we had also removed the eps genes prior to examining the adhesive systems of our functional cellular glues. EPSs are commonly negatively charged [50], and it is known that electrostatic interactions between polyanions and polycations (along with metal ions) contribute to the strong underwater adhesion of sandcastle worms [29]. Lap shear tests revealed that biofilms produced by a strain containing the eps genes had significantly increased shear adhesive strength compared to the bslA⁻ tasA⁻ eps⁻ biofilms; further, we observed increased shear adhesion when we cured EPS-containing biofilms via treatment with Fe³⁺ ions (FIG. 22). Scanning electron microscopy (SEM) revealed that this curing treatment condensed the biofilm matrix, and EDS analysis showed that these biofilms had overlapped mapping signals for Fe and P elements (FIG. 23), which was in agreement with the characteristic ATR-FTIR peaks of P═O (1117 cm⁻¹) and PO₃²⁻ (979 cm⁻¹, 1084 cm⁻¹, 1264 cm⁻¹) groups that only appeared in EPS-containing biofilms (FIG. 24). Collectively, these results indicate that the increased adhesion observed for the EPS-containing iron-cured biofilms resulted from electrostatic interactions between Fe³⁺ ions and the PO₃²⁻ groups present in EPS.

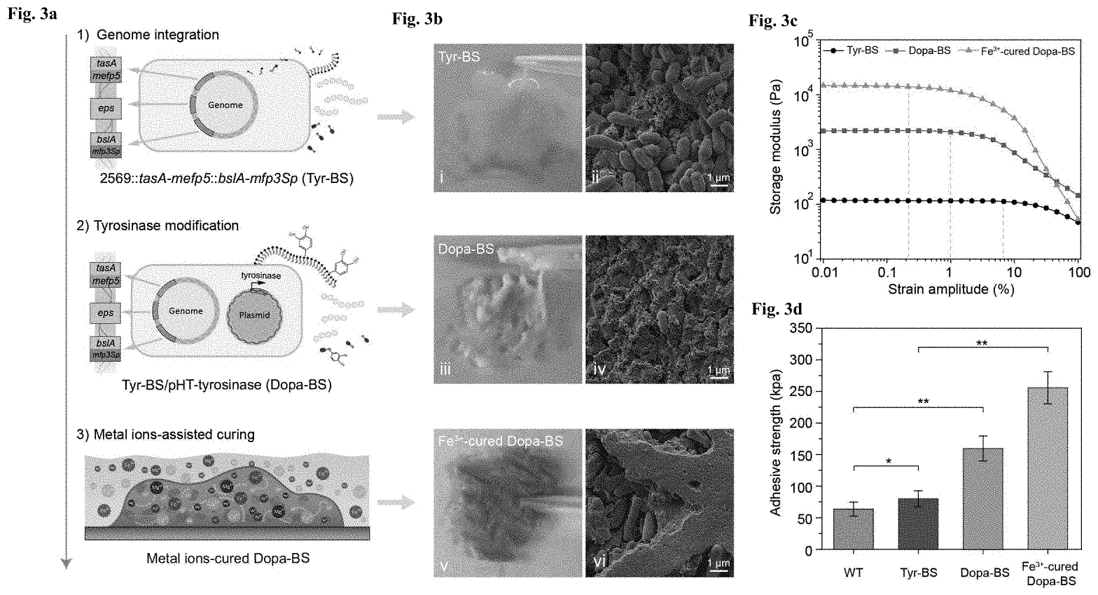

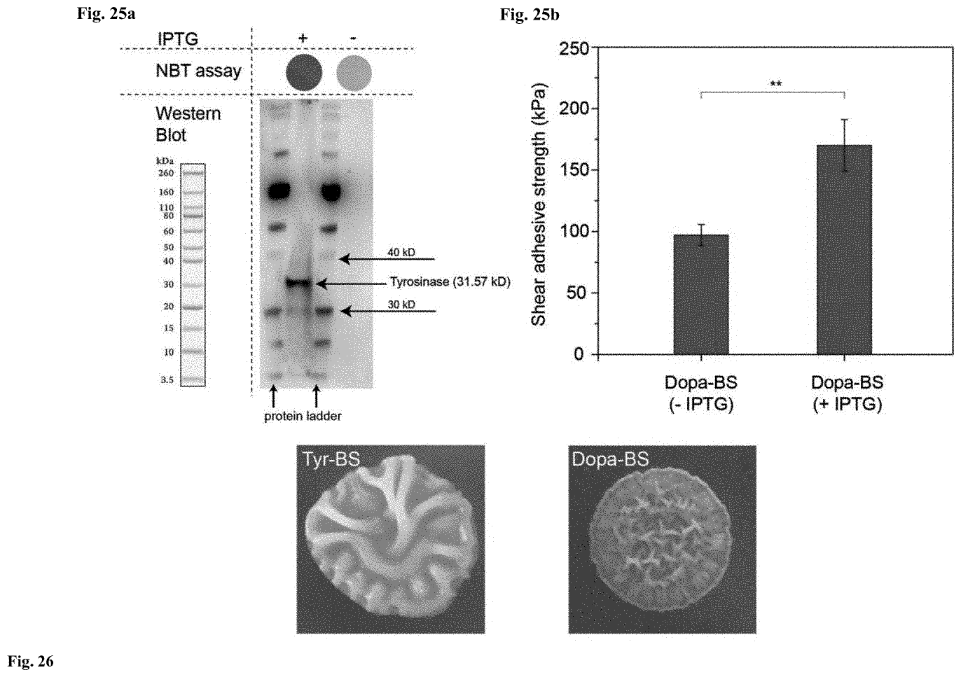

Having examined the three main constituent components of our functional living glue concept individually, we next conducted a series of successive experiments in which we initially integrated the disparate components, subsequently elaborated the system via the inducible expression of a tyrosine-modifying enzyme (tyrosinase), and finally cured the biofilms via the addition of a variety of metal ions (FIG. 3a). First, these experiments examined a B. subtilis strain whose genome contained the native eps genes; in this strain, the native tasA locus had been replaced with the construct for the TasA-Mefp5 fusion protein and the native b1sA locus had been replaced with the construct encoding the BslA-Mfp3Sp fusion protein (denoted as the integrated B. subtilis (Tyr-BS) strain). Second, we transformed the Tyr-BS strain with a plasmid expressing a tyrosinase (denoted as the modified B. subtilis (Dopa-BS) strain). Our rationale here is that the activity of tyrosinase dramatically increases the adhesion of tyrosine-rich mussel foot proteins, such as Mefp-3 and Mefp-5 in Mytilus edulis [32, 51, 52]. Specifically, tyrosinase catalyzes the hydroxylation of tyrosine residues to form Dopa on these proteins, and once these modified proteins are secreted, the DOPA groups can form bidentate hydrogen bonds with surfaces and can also be extensively crosslinked via the oxidation of Dopa to Dopa-quinone, among other biofilm components [10, 13] (FIG. 25). Third, we incorporated a variety of different metal ions (e.g. Ca²⁺, Mg²⁺, Fe³⁺) into biofilms produced by the Dopa-BS strain to facilitate a curing process somewhat similar to the aforementioned treatment of the EPS with Fe³⁺. It is important to note that beyond the roles of Dopa in hydrogen bond formation, hydrophobic interactions and cation-π interactions, Dopa-quinone groups in the biofilm matrix are also thought to form strong crosslinks with crosslinking partners including Dopa and other residues in biofilms (e.g., histidine, lysine, and cysteine) [53-55], while the Dopa groups might also contribute to chelating Fe³⁺ ions present in biofilms via the formation of Dopa-Fe³⁺ coordination complexes [16, 20].

Visual examination clearly indicated that the Dopa-BS biofilm was more tightly integrated and had a more wrinkled morphology than the Tyr-BS biofilm (FIG. 26). When agitated with a pipette tip, the Tyr-BS biofilm behaved like a soft gel, and the Fe³⁺-cured Dopa-BS biofilm resembled a cement-like solid, with the Dopa-BS biofilm being intermediate between these two in consistency (FIG. 3b). At the microscopic scale, scanning electron microscopy (SEM) revealed the same trend, with the Dopa-BS biofilm having a much more compact matrix structure and the Fe³⁺-cured Dopa-BS biofilm appearing as a cement-like aggregate (FIG. 3b). In accord with these visual observations, rheological measurements indicated that Dopa-BS biofilm was stiffer and more viscous than the Tyr-BS biofilm and that the Fe³⁺-cured Dopa-BS biofilm was stiffer than the uncured Dopa-BS biofilm (FIG. 3c and S22-24). Note that the viscosity of all engineered biofilm glues generally showed a linear decrease as a function of the shear rate. These results indicate that our engineered biofilms do not exhibit any obvious shear-thinning or shear-thickening responses over a wide range of shear rates.

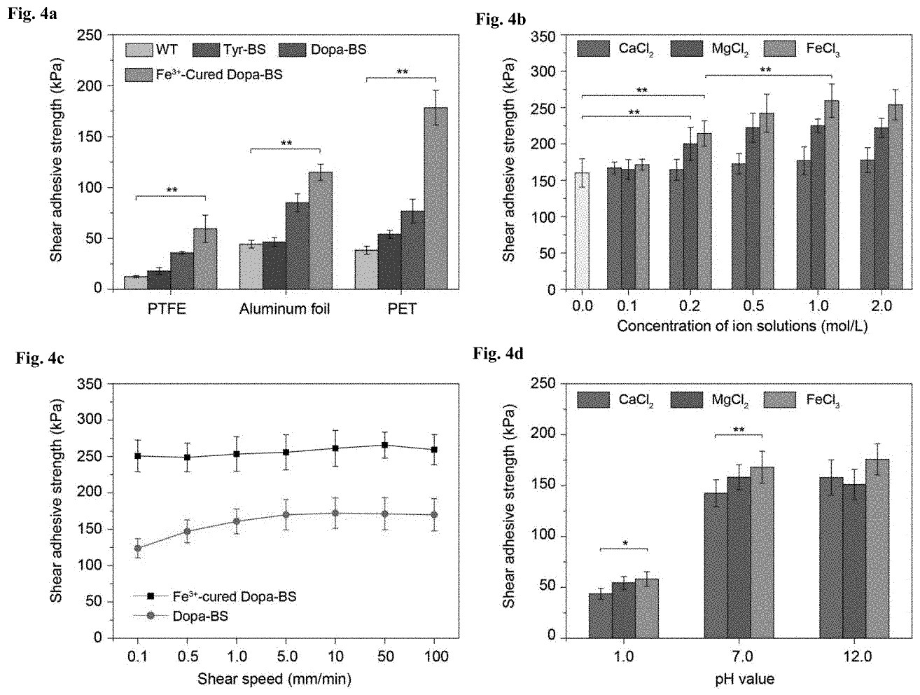

Lap shear tests showed dramatic increases in shear adhesive strength resulting from the successive engineering and curing steps we undertook during the development of our functional cellular glues (FIG. 3d). Specifically, we observed a significant increase in shear adhesive strength between an unaltered wild-type B. subtilis biofilm and the Tyr-BS biofilm, which might arise from cation-π and electrostatic interactions within the fused tyrosine-rich Mefp5 and Mfp3Sp domains [15, 44]. There was also a large increase in adhesive strength between the Tyr-BS biofilm and the tyrosinase-expressing Dopa-BS biofilm. The increased strength likely resulted from the covalent crosslinking between Dopa-quinone and other functional residues as described above. The highest shear adhesive strength we observed was the Fe³⁺-cured Dopa-BS biofilm, which was 1.6 times of the adhesive strength of the uncured Dopa-BS biofilm and 4-fold higher than the wild-type B. subtilis biofilm (FIG. 3d). Similar to the results using stainless steel sheet as test substrate, shear adhesion performance of the four different biofilms assessed with a variety of other substrates (e.g. polytetrafluoroethylene (PTFE), aluminum foil, and polyethylene terephthalate (PET) followed the same trend: Fe³⁺-cured Dopa-BS>Dopa-BS>Tyr-BS>wild type biofilms (FIG. 4a).

We next assessed the shear adhesion performance of the metal ion-cured Dopa-BS biofilms under a wide range of test conditions (ion type, ion concentration and shear speed). The addition of Mg²⁺ and Fe³⁺, even at 0.2 mol/L, all caused significant increases in shear adhesive strength compared to the uncured Dopa-BS biofilms. While further increase in Ca²⁺ ion concentration didn't have apparent effect, the increase of magnesium or iron ion concentrations further increased the shear adhesive strength of biofilms until their respective maximum adhesion strengths reached at concentrations ≥0.5 mol/L (FIG. 4b). Interestingly, at relatively higher concentration (≥0.2 mol/L), the Fe³⁺-cured Dopa-BS biofilms always exhibited the largest shear adhesive strength, with Mg²⁺ being the intermediate (FIG. 4b). Moreover, compared to the slight increase in shear adhesion strength of the Dopa-BS as a function of shear speed, the shear adhesive strength of the Fe³⁺-cured Dopa-BS biofilm almost remained unchanged across a wide range of tested shear speeds, possibly due to the formation of concrete-like structures that prevent potential dissipation of viscosity as compared to the Dopa-BS biofilm (FIG. 4c) [56]. In addition, the shear adhesive strength of the metal ion-cured Dopa-BS biofilms all decreased after exposure to acidic solutions (pH 1.0), potentially owing to protonation of the carboxyl and phosphate functional groups and the change of coordinative cross-linking between metal ions and Dopa (for example, crosslinking tris, bis with Fe³⁺ forming at pH>7.0, and mono-Fe³⁺ forming at lower pH value) in the biofilm network (FIG. 4d).

To assess the adhesive peel strength of the engineered biofilm glues with different substrates, we applied a 90° Peeling Test. We chose the elastic Dopa-BS biofilms here because they are more deformable compared to cured ones, thus forming more regular surfaces that are more amenable for the 90° Peeling Test. We found that Dopa-BS exhibited higher adhesion on the aluminum (Al) and polyethylene terephthalate (PET) surfaces than on the glass surface, possibly due to a stronger interfacial adhesion via metal-coordination interactions or Dopa-mediated hydrophobic interactions generated between the Dopa-BS biofilm matrix and the Al/PET surfaces (FIG. 30).

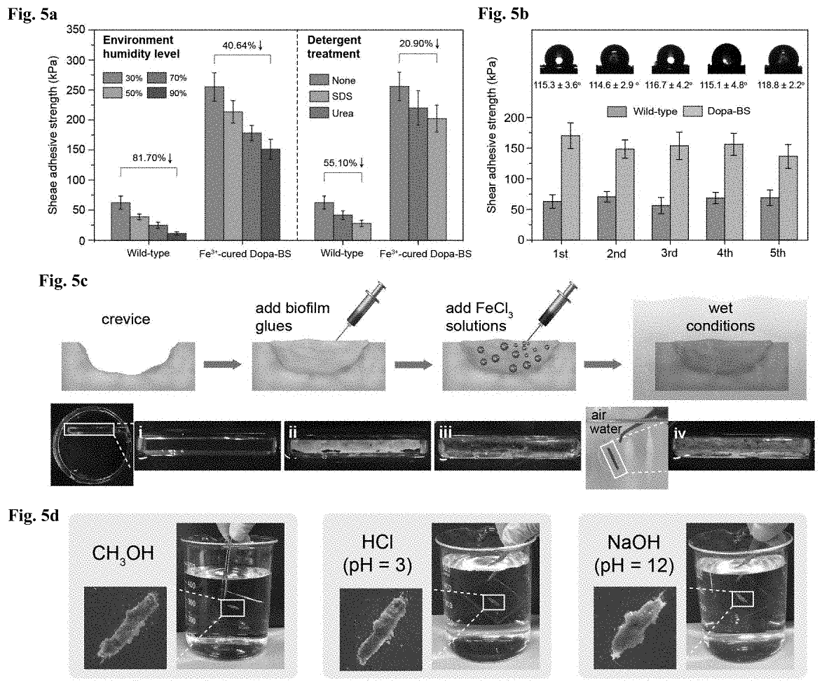

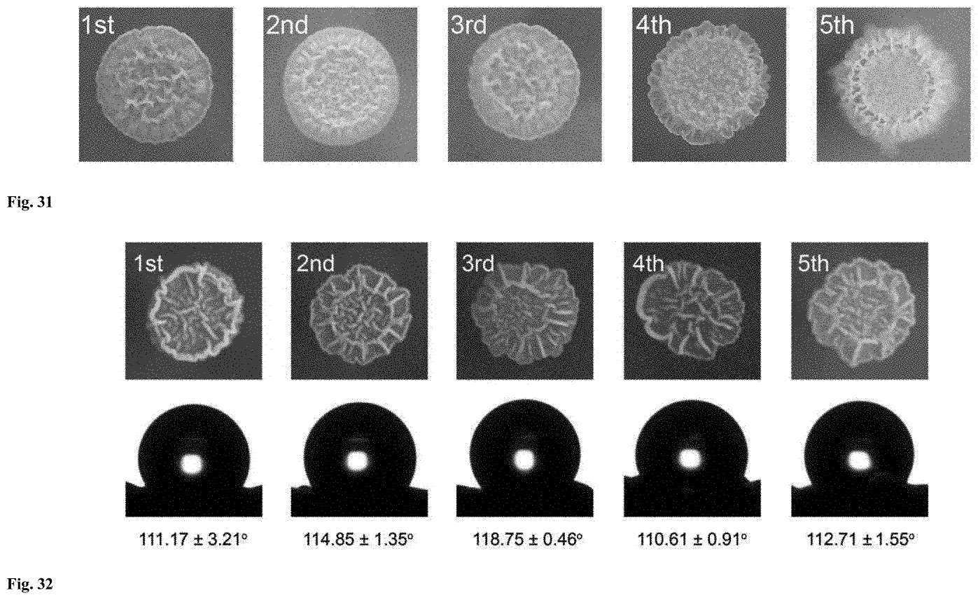

We next tested the shear adhesive strength of the best-performing biofilm with a variety of environmental challenges. We examined its resistance to moisture by analyzing shear adhesive strength in lap shear tests conducted at a variety of humidity levels and found that the Fe³⁺-cured Dopa-BS biofilm consistently and dramatically outperformed the wild-type B. subtilis biofilm; in fact, even at 90% relative humidity, the Fe³⁺-cured Dopa-BS biofilms retained 60% of their shear adhesive strength (FIG. 5a). Lap shear tests conducted in the presence of detergents also highlighted the strong adhesion performance of the Fe³⁺-cured Dopa-BS biofilm in challenging conditions: The Fe³⁺-cured Dopa-BS biofilm retained 80% of their shear adhesive strength after treatment with SDS detergent (FIG. 5a). One of the most attractive attributes of living materials is their ability to self-regenerate, and we tested this with our Dopa-BS biofilms by scraping the cultures and passaging them through five successive cycles of re-inoculation and growth on agar plates (FIG. 31). No obvious morphological changes were detected among the regenerated biofilms after the regeneration process. We further used water contact angle assays and lap shear measurements to examine if any changes in hydrophobicity (an indication of the general physicochemical properties) and adhesive capacity of the biofilms occurred during the regeneration process. Like the wild-type biofilm, both the hydrophobicity and shear adhesive strength of the Dopa-BS biofilm of each generation did not change significantly after repeated passaging (FIGS. 5b and 31-32).

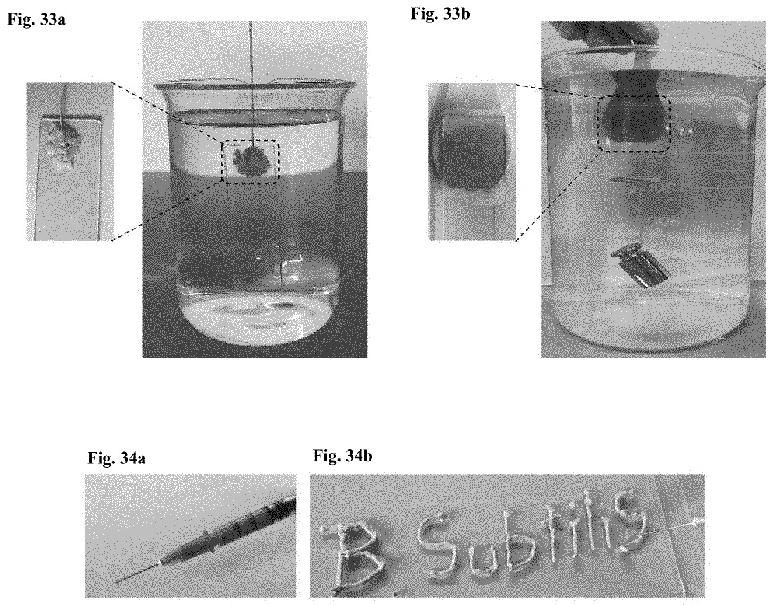

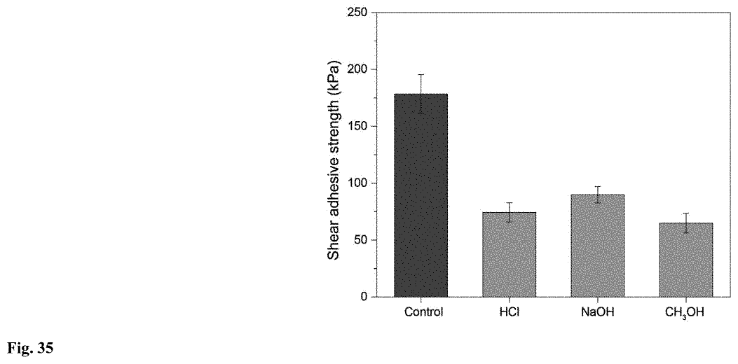

We next turned to test the use of our functional cellular glues for practical applications (FIGS. 5c and 33). We used the Dopa-BS biofilm as injectable living materials to fill crevices on a substrate made of PDMS, a material known to resist most adhesives. Owing to its gel-like properties, the Dopa-BS biofilm could easily be injected into crevices, and we injected Fe³⁺ ions in solution to cure the Dopa-BS biofilm in the crevices (FIGS. 5c and 34). These injected living materials and ions cured spontaneously, turning into a cement-like solid that completely filled the gaps within 5 min Notably, the resulting cement-like solid maintained its shape and geometry, even when it was subjected to harsh conditions over night, including immersion in solutions across a wide range of pH values and exposure to organic solvents (FIG. 5d). The shear adhesive strength of Fe³⁺-cured Dopa-BS biofilm glues was further investigated after these treatments. Nearly 36.4% and 42.7% of the shear adhesive strength could be retained even after 10 h exposure in CH₃OH and HCl solution respectively, implying their relatively strong tolerance towards harsh conditions (FIG. 35).

We here developed and demonstrated the concept of functional cellular glues with highly engineerable biofilms. Our ‘living biofilm glues’ represent a new class of adhesives that for the first time possess the distinctive “living” attributes that are beyond the reach of the vast majority of existing synthetic adhesives. The adhesive strength of these living systems can be further increased, for example by rationally selecting adhesive compositions and optimizing protein expression, or using directed evolution methods. Indeed, researchers can integrate additional genetically encoded adhesive components to the editable genomes of these glues to confer additional (e.g. environmentally responsive and tunable) functional elaborations to further improve their adhesive performance and utility in biomedical and industrial contexts. In addition, these cellular glues are alive and thus evolvable, so directed evolution methods via targeted or random mutations can be used to further optimize their performance for targeted indications and applications.

Our biofilms provide an engineering platform of smart living glues with previously unattainable functions. For instance, corrosion-sensitive materials can be coated with smart biofilm glues that can use bio-sensors to detect damage signals that trigger the expression and secretion of reparative adhesives. Moreover, considering that B. subtilis is a ‘generally regarded as safe’ (GRAS) organism, probiotic applications of our cellular glues include ingested cells can form a biofilm bandage over a wound in a targeted area and locally secrete a therapeutic agent on demand.

### REFERENCES

1. Dolgin, E., Nat Med (2013) 19 (2), 124
2. Lee, B. P., et al., Annu Rev Mater Res (2011) 41, 99
3. Baik, S., et al., Adv Sci (2018) 5 (8)
4. Li, J., et al., Science (2017) 357 (6349), 378
5. Lang, N., et al., Sci Transl Med (2014) 6 (218)
6. Ryou, M. H., et al., Adv Mater (2011) 23 (27), 3066
7. Jing, X., et al., Acs Appl Mater Inter (2018) 10 (24), 20897
8. Ku, S. H., et al., Langmuir (2010) 26 (19), 15104
9. Kang, S. M., et al., Angew Chem Int Edit (2010) 49 (49), 9401
10. Waite, J. H., The Journal of Experimental Biology (2017) 220 (4), 517
11. Kamino, K., Marine Biotechnology (2008) 10 (2), 111
12. Stewart, R. J., et al., Adv Colloid Interfac (2011) 167 (1-2), 85
13. Hofman, A. H., et al., Adv Mater (2018) 30 (19)
14. Stewart, R. J., et al., J Polym Sci Pol Phys (2011) 49 (11), 757
15. Kim, S., et al., P Natl Acad Sci USA (2016) 113 (7), E847
16. Harrington, M. J., et al., Science (2010) 328 (5975), 216
17. Ahn, B. K., J Am Chem Soc (2017) 139 (30), 10166
18. Meredith, H. J., et al., Adv Funct Mater (2014) 24 (21), 3259
19. Lee, H., et al., Science (2007) 318 (5849), 426
20. Maier, G. P., et al., Science (2015) 349 (6248), 628
21. Zhao, Q., et al., Nature Materials (2016) 15 (4), 407
22. Kim, S., et al., Acs Nano (2017) 11 (7), 6764
23. Chipara, A. C., et al., Materials Today Chemistry (2018) 9, 149
24. Barlow, D. E., et al., Langmuir (2010) 26 (9), 6549
25. Zhong, C., et al., Nat Nanotechnol (2014) 9 (10), 858
26. Cui, M. K., et al., Apl Mater (2017) 5 (11)
27. Petrone, L., et al., Nature communications (2015) 6
28. Priemel, T., et al., Nature Communications (2017) 8
29. Stewart, R. J., et al., J Exp Biol (2004) 207 (26), 4727
30. Jonker, J. L., et al., J Morphol (2012) 273 (12), 1377
31. Nakano, M., and Kamino, K., Biochemistry-Us (2015) 54 (3), 826
32. Lee, H., et al., PNAS (USA) (2006) 103 (35), 12999
33. Wei, W., et al., Adv Funct Mater (2016) 26 (20), 3496
34. Harwood, C. R., Trends in Biotechnology (1992) 10 (7), 247
35. Jiaofang Huang, et al., Nature Chemical Biology (2018)
36. Romero, D., et al., PNAS (USA) (2010) 107 (5), 2230
37. Kobayashi, K., and Iwano, M., Molecular Microbiology (2012) 85 (1), 51
38. Vlamakis, H., et al., Nat Rev Microbiol (2013) 11 (3), 157
39. Wang, X. Y., et al., Adv Mater (2018) 30 (16)
40. Wang, Y. Y., et al., J Mol Biol (2018) 430 (20), 3720
41. Wincing, J. N., et al., MRS bulletin (2011) 36 (05), 385
42. Maxwell, B., and Guimon, C., Journal of Applied Polymer Science (1962) 6 (19), 83
43. Lembre, P., et al., Biofouling (2014) 30 (4), 415
44. Gebbie, M. A., et al., Nat Chem (2017) 9 (5), 473
45. Lu, Q. Y., et al., J R Soc Interface (2013) 10 (79)
46. Wei, W., et al., Acta Biomater (2014) 10 (4), 1663
47. Hobley, L., et al., P Natl Acad Sci USA (2013) 110 (33), 13600
48. Keeble, A. H., et al., Angew Chem Int Edit (2017) 56 (52), 16521
49. Kesel, S., et al., Applied and environmental microbiology (2016) 82 (8), 2424
50. Flemming, H. C., and Wingender, J., Nat Rev Microbiol (2010) 8 (9), 623
51. Yu, J., et al., P Natl Acad Sci USA (2013) 110 (39), 15680
52. Lin, Q., et al., P Natl Acad Sci USA (2007) 104 (10), 3782
53. Zhao, H., and Waite, J. H., J Biol Chem (2006) 281 (36), 26150
54. McDowell, L. M., et al., J Biol Chem (1999) 274 (29), 20293
55. Miserez, A., et al., J Biol Chem (2010) 285 (49), 38115
56. Labonte, D., and Federle, W., Soft Matter (2015) 11 (44), 8661

Strain and Plasmid Construction: Construction of B. subtilis bslA⁻, bslA⁻ tasA⁻, and bslA⁻ tasA⁻ eps⁻ Mutants.

To create biofilm-defective strains, we first constructed three suicide plasmids using the pMAD plasmid vector: pMAD-DbslA, pMAD-DtasA, and pMAD-Deps. To construct the pMAD-DbslA suicide plasmid, the primer pair Db-up-F/R was used to amplify the ˜1 kb fragment from the wild-type genome upstream of bslA and the primer pair Db-down-F/R was used to amplify the ˜1 kb fragment from the wild-type genome downstream of the bslA gene. The two bslA flanking regions were fused together into a 2 kb fragment, then inserted into pMAD linearized by SmaI/EcoRI digestion using a Gibson Assembly Kit and the respective restriction endonucleases (New England Biolabs) to obtain the suicide plasmid pMAD-DbslA that targeted the bslA gene for deletion.

To construct pMAD-DtasA suicide plasmid, primer pairs of Dt-up-F/R and Dt-down-FIR were used to amplify the 1 kb fragments on the 5′ and 3′ flanking regions of the tasA gene, respectively. The PCR products were then fused together into a 2 kb fragment (for targeted deletion of the tasA gene), which was then inserted into SmaI/NcoI linearized pMAD to create the suicide plasmid pMAD-DtasA.

Construction of the pMAD-Deps plasmid, for targeted deletion of the eps gene cluster, was conducted according to the same method as above except using primer pairs of De-up F/R and De-down F/R to amplify the respective flanking regions of the eps genes, and BamHI/SalI restriction sites for insertion into pMAD.

The suicide plasmid pMAD-DbslA was then transformed into B. subtilis wild-type 2569 competent cells based on the chemical transformation method (Spizizen method)[1]. The colonies were then selected on LB agar plate supplemented with 5 μg/mL erythromycin at 30° C. Transformants were restreaked onto non-selective LB and grown overnight at 42° C. Isolates were patched onto LB erythromycin agar plate to counter select for cells that had lost the plasmid. PCR fragments from the genomic DNA of mutants were sequenced to confirm the deletion of bslA. The resultant mutant strain was referred to as B. subtilis bslA⁻ mutant strain.

Similarly, B. subtilis bslA⁻ tasA⁻ mutant strain was created by transformation of the pMAD-DtasA into the above bslA⁻ strain following the selection protocol described above, while the B. subtilis bslA⁻ tasA⁻ eps⁻ mutant strain was obtained by further transformation, selection and subsequent counterselection of the pMAD-Deps plasmid into the bslA⁻ tasA⁻ strain.

Construction of B. subtilis 2569::tasA-Mefp5::bslA-mfp3Sp Integration Strain.

To create a genome integration strain, we constructed pMAD-tasA-mefp5 and pMAD-bslA-mfp3Sp suicide plasmids. For pMAD-tasA-mefp5, Tm5-up-F/R and Tm5-down-F/R primers were used to amplify the ˜1 kb regions flanking either side of tasA gene, respectively. While primer pairs Tm5-F/R were used to amplify tapA-sipW-tasA-mefp5 fragment, the upstream flank, tapA-sipW-tasA-mefp5 fusion, and downstream flank were assembled in that order and ligated into pMAD by SmaI/NcoI digestion using a Gibson Assembly Kit to obtain the pMAD-tasA-mefp5 plasmid.

The pMAD-bslA-mfp3Sp was constructed using the same method as above, except with B3Sp-up-FIR and B3Sp-down-FIR primers to amplify bslA 1 kb flanking regions, and the primer pair B3Sp-FIR to amplify the bslA-mfp3Sp fragment. The upstream region, bslA-mfp3Sp insert, and downstream fragments were fused in that order and inserted into SmaI/NcoI digested pMAD also using a Gibson assembly kit to obtain the final integration plasmid.

Afterwards, the plasmid pMAD-tasA-mefp5 was transformed into B. subtilis wild-type 2569 competent cells using the same procedure as before. The strain were then selected following the same methods for the genome mutation described above to achieve a transitional bacteria strain named 2569::tasA-mefp5. The plasmid pMAD-bslA-mfp3Sp was then transformed into the 2569:: tasA-mefp5 competent cell. The created strain was then selected using the same method to obtain the final integration strain 2569::tasA-mefp5::bslA-mfp3Sp (denoted as integrated B. subtilis strain “Tyr-BS”).

Construction of Protein Expression Plasmids.

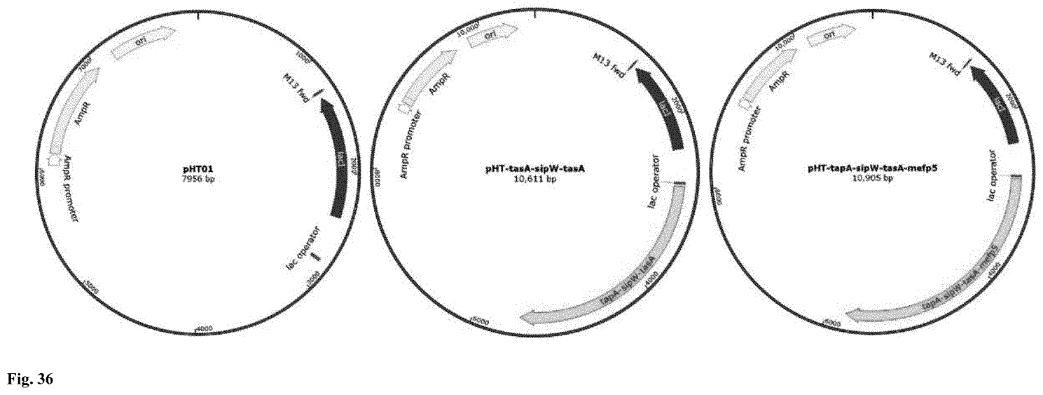

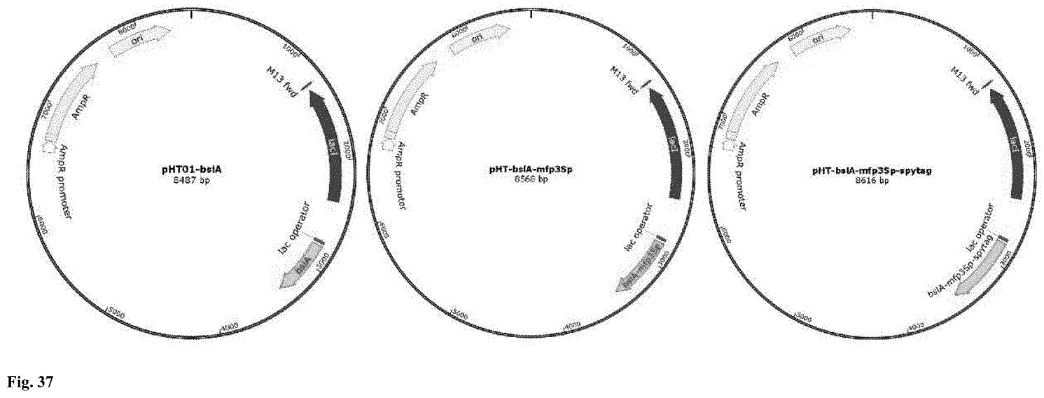

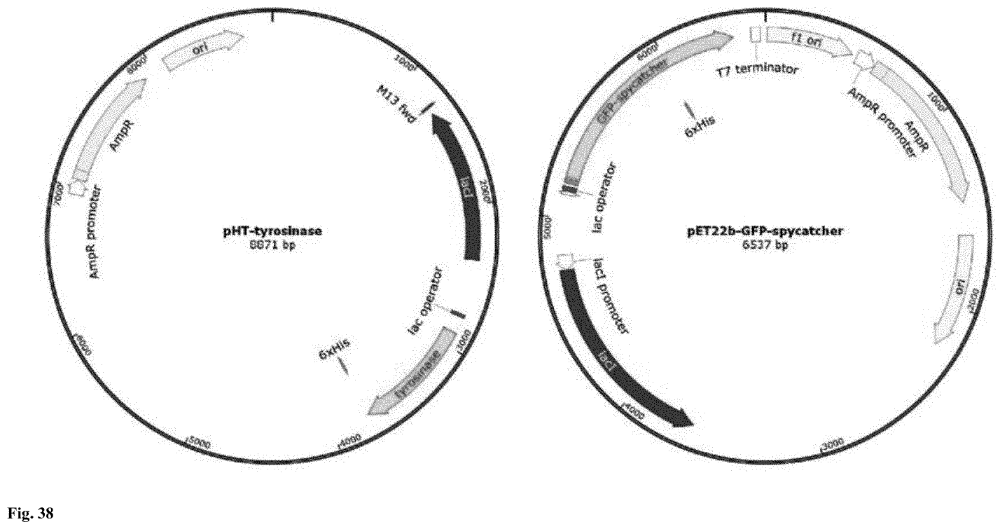

The plasmids used in this study for the expression of TasA, TasA-Mefp5, BslA, BslA-Mfp3Sp, BslA-Mfp3Sp-Spytag, and Tyrosinase proteins were constructed. The gene fragments for tapA-sipW-tasA, tapA-sipW-tasA-mefp5, bslA, bslA-mfp3Sp, bslA-mfp3Sp-spytag and tyrosinase were synthesized by Genewiz and amplified by primer pairs of pTasA-F/R, pTasA-F/pTasA-mefp5-R, pBslA-F/R, pBslA-F/pBslA-mfp3Sp-R, pBslA-F/pBslA-mfp3Sp-spytag-R and pTyro-F/R, respectively. These gene fragments were then inserted into the pHT01 plasmid at BamHI/SmaI sites to create plasmids pHT-tapA-sipW-tasA, pHT-tapA-sipW-tasA-mefp5, pHT-bslA, pHT-bslA-mfp3Sp, pHT-bslA-mfp3Sp-spytag and pHT-tyrosinase, respectively. The gene fragment for tapA-sipW-mefp5 was also amplified by the primer pair of pTyr-tm5-F/R and then inserted into the pHT-tyrosinase at SmaI site to create the plasmid pHT-tyr-tasA-mefp5. These created plasmids were correspondingly applied for protein expression of TasA, TasA-Mefp5, BslA, BslA-Mfp3Sp, BslA-Mfp3Sp-Spytag, Tyrosinase, and both Tyrosinase and TasA-Mefp5 respectively under biofilm culture conditions when transformed into B. subtilis strains via the Spizizen transformation method mentioned above. Plasmid maps were shown in FIGS. 36-38.

The genes encoding GFP-Spycatcher and mCherry-Spycatcher were synthesized and inserted into pET22b plasmid at NedI/XhoI sites by Genewiz to create the pET22b-GFP-spycatcher and pET22b-mCherry-spycatcher plasmids.

Biofilm Culture Conditions.

LuriaBertani (LB) broth: 1% tryptone (Difco), 0.5% yeast extract (Difco), 0.5% NaCl. MSgg broth: 100 mM morpholine propane sulphonic acid (Mops) (pH 7), 0.5% glycerol, 0.5% glutamate, 5 mM potassium phosphate (pH 7), 50 μg/mL tryptophan, 50 μg/mL phenylalanine, 2 mM MgCl₂, 700 μM CaCl₂, 50 μM FeCl3, 50 μM MnCl₂, 2 μM thiamine, 1 μM ZnCl₂. To prepare LB and MSgg solid plates, corresponding LB and MSgg solution supplemented with 1.5% agar were solidified upon cooling.

Host B. subtilis strains without antibiotic resistance (bslA⁻ tasA⁻ eps⁻, bslA⁻, bslA⁻ tasA⁻, and Tyr-BS) were streaked from frozen glycerol stocks and grown on LB plates overnight at 37° C. Seed cultures were grown in LB medium at 37° C. by inoculating monoclonal in LB plate. Seed cultures were subsequently re-inoculated to LB broth at a final cell density of 5×10⁷ cells/mL and grown at 37° C. for 3 h. Cell pellets were then collected through centrifugation (to remove residual LB medium) and resuspended in ddH₂O at a cell density of 5×10⁷ cells/mL. For solid-plate biofilm formation, 2.5 μL of cell suspension was dropped onto MSgg plates and then grown at 30° C. for 2 days. For liquid biofilm formation, 40 μL of resuspended cells were added into 4 mL MSgg liquid culture and then statically cultured at 30° C. for 48 hours.

Host strains harboring pHT plasmids bslA⁻ tasA⁻ eps⁻/pHT-tapA-sipW-tasA (denoted as bslA⁻ tasA⁻ eps⁻/TasA); bslA⁻ tasA⁻ eps⁻/pHT-tasA-sipW-tasA-mefp5 (denoted as bslA⁻ tasA⁻ eps⁻/TasA-Mefp5); bslA⁻/pHT-bslA (denoted as bslA⁻/BslA); bslA⁻/pHT-bslA-mfp3Sp-spytag (denoted as bslA⁻/BslA-Mfp3Sp-Spytag); and bslA⁻/pHT-bslA-mfp3Sp (denoted as bslA⁻/BslA-Mfp3Sp)), were grown in LB medium supplemented with 5 μg/mL chloramphenicol during all inoculation processes. For biofilm formation, the final MSgg liquid or solid culture medium was supplemented with 5 μg/mL chloramphenicol and 1 mM IPTG (for the induction of protein expression). To culture biofilm formed by the host strain containing pHT-tyrosinase plasmid (Tyr-BS/pHT-tyrosinase, denoted as modified B. subtilis “Dopa-BS”), MSgg medium for final biofilm information was also added with 0.4 μg/mL CuSO₄ to ensure full bioactivity of tyrosinase. Biofilm formation for host strain harboring plasmid bslA⁻ tasA⁻ eps⁻/pHT-tyrosinase-tasA-mefp5 (denoted as bslA⁻ tasA⁻ eps⁻/Tyrosinase-TasA-Mefp5) was cultured following the same protocol as described for the Dopa-BS strain.

Contact angle measurement. We used contact angle measurement to detect the hydrophobicity of the bslA⁻ biofilm upon the expression of the BslA and BslA-Mfp3Sp proteins, and also used these assays to detect the ‘similarity’ amongst successive generations of the “Dopa-BS” biofilms. The contact angle measurement was performed with ddH₂O (2 μL) at room temperature using Theta Lite (Biolin) following the sessile drop method [2]. Isolated biofilm samples were first prepared by carefully cutting MSgg solid plate to ensure an undamaged surface of biofilms and then placed onto a glass slide horizontally. The contact angle was then analyzed using One Attension software.

Congo red (CR) quantitative assay. To quantify the amyloid components of TasA and TasA-Mefp5 proteins produced in the bslA⁻ tasA⁻ eps⁻/TasA and bslA⁻ tasA⁻ eps⁻/TasA-Mefp5 biofilms, respectively, biofilms collected from a 4-mL liquid MSgg medium culture were resuspended in 1 mL PBS buffer in a microcentrifuge tube. 100 μL 2.5 mg/mL CR solution was added to 1 mL of the mixed solution. The mixed solution was kept for 30 mM to ensure thorough binding between CR and amyloid components at room temperature and centrifuged at 5000 g for 10 mM Concentrations of CR in the supernatant and in the mixed solution (before centrifugation) were quantified by the absorbance at a range of wavelength from 400 to 600 nm using CYTATION (BioTek). The amount of CR absorbed by the biofilm samples was quantified by subtracting the supernatant absorption from the mixed solution absorptions following the same approach in a previous study [2]. Results are presented in FIG. 10.

Nitro Blue Tetrazolium (NBT) Assays.

To confirm the enzymatic modification of tyrosine residues into Dopa in the biofilm matrix with tyrosinase expressed in vivo, an NBT assay was applied. Specifically, Dopa-BS biofilms grown with IPTG induced and uninduced tyrosinase expression from 4 mL MSgg liquid medium were collected by centrifugation at 5000 g for 5 min and resuspended in 1 mL PBS buffer. The mixed solutions were then adjusted to an initial cell density of 5×10⁷ cells/mL. For NBT assays, 100 μL solutions were spotted onto nitrocellulose membranes with a dot blot manifold (Schleicher & Schuell Minifold-I Dot-Blot System) as described in a previous study[3]. The membranes containing biofilm solutions were then incubated in 30 mL fresh 0.6 mg/mL NBT solution in 2 M potassium glycinate buffer (pH=10.0) at room temperature in the dark for 1 h. Afterwards, the membranes were washed with 30 mL 0.16 M sodium borate solution twice and soaked in another 20 mL sodium borate solution overnight. Images of the stained membranes were taken with a scanner. The results are presented in FIG. 25a.

Atomic Force Microscopy (AFM).

AFM methodology was applied to detect the formation of extracellular nanofibres in the bslA⁻ tasA⁻ eps⁻ biofilms containing TasA or TasA-Mefp5 proteins. For AFM sample preparation, 50 μL bslA⁻ tasA⁻ eps⁻, bslA⁻ tasA⁻ eps⁻/TasA, or bslA⁻ tasA⁻ eps⁻/TasA-Mefp5 biofilm-containing solution was carefully taken from the MSgg liquid medium by a pipette and spotted onto a mica plate (1×1 cm). The excessive liquid biofilm solution was then blotted off using a filter paper (Whatman no. 1) and blow-dried using nitrogen flow. The sample was then imaged by a Bruker Dimension Fastscan AFM on tapping mode using Veecoprobes Sb-doped Si cantilevers (ρ=0.01-0.025 Ω-cm, k=40 N/m, v 300 kHz). Results were shown in FIG. 6.

Transmission Electron Microscopy (TEM) and Energy Dispersive X-Ray Spectroscopy (EDS).

TEM methodology was applied to confirm if extracellular amyloid nanofibres were assembled around cell surfaces in the bslA⁻ tasA⁻ eps⁻, bslA⁻ tasA⁻ eps⁻/TasA and bslA⁻ tasA⁻ eps⁻/TasA-Mefp5 biofilm samples.

For TEM sample preparation, 10 μL biofilm-containing solution was carefully taken from the MSgg medium by a pipette and spotted onto carbon-coated TEM grids (Zhongjingkeyi Technology, EM Sciences) for 5 min. The grids were then washed by 20 μL PBS buffer and 20 μL ddH₂O followed by blotting off the excess solution on a filter paper (Whatman no. 1).

For immune-localization of TasA proteins, TEM grids with biofilm samples were floated on 20 μL blocking buffer (PBS buffer containing 1% skim milk and 0.1% Tween 20) for 30 min, followed by incubation for 2 h in 20 μL droplet of blocking buffer with anti-TasA primary antibody diluted at 1:150 ratio. Afterwards, the samples were rinsed 3 times in 20 μL PBST (PBS buffer containing 0.1% Tween 20) and subsequently transferred to 20 μL droplet of blocking buffer with goat anti-rabbit secondary antibody conjugated to 20-nm gold particles diluted at 1:5000 ratio (EM.GAR20, BBI), where it was incubated for 1 h. The grids were then washed with 20 μL PBS buffer and 20 μL ddH₂O. The excess liquid was blotted off on a filter paper (Whatman no. 1) and the sample was stained with 20 μL uranyl acetate (1-2% aqueous solution). The air-dried samples were eventually examined in a JSM 1400 transmission electron microscope at an accelerating voltage of 120 kV. The immuno gold nanoparticles were further characterized by high resolution TEM (HRTEM) and corresponding EDS mapping in a JEM-F200 electron microscope at an accelerating voltage of 300 kV.

To detect the full-length TasA-Mefp5 fusion protein (with a 6× Histidine at its C-terminal), the bslA⁻ tasA⁻ eps⁻/TasA-Mefp5 (His-tagged) strain was cultured in a 4 mL biofilm forming MSgg culture medium, supplemented with 50 μL nitrilotriacetic acid (NTA)-decorated gold nanoparticles solution following the same method in previous studies [2]. Biofilms sample derived from bslA⁻ tasA⁻ eps⁻/TasA or bslA⁻ tasA⁻ eps⁻/TasA-Mefp5 strain was applied as negative controls. For TEM sample preparation, 10 μL biofilm-containing solution was carefully taken from the MSgg medium and spotted onto carbon-coated TEM grids (Zhongjingkeyi Technology, EM Sciences) for 5 min. The grids were then washed with 20 μL PBS buffer. The excess liquid was blotted off on a filter paper (Whatman no. 1) and the sample was stained with 20 μL uranyl acetate (1-2% aqueous solution). The air-dried samples were eventually examined in a JSM 1400 transmission electron microscope at an accelerating voltage of 120 kV.

Scanning Electron Microscopy (SEM) and Energy Dispersive X-Ray Spectroscopy (EDS).

Morphologies of biofilms samples were acquired with scanning electron microscopy. For SEM sample preparation, the bslA⁻ tasA⁻, Tyr-BS and Dopa-BS biofilms were scraped from MSgg culture plates with a final weight around 0.5 g, respectively. The corresponding sample was then transferred onto an aluminum foil. The Fe³⁺-cured Dopa-BS and bslA⁻ tasA⁻ biofilms was prepared by mixing 5 μL 1M FeCl₃ solution with the biofilms matrix under gentle agitation.

Afterwards, all samples (Tyr-BS, Dopa-BS, and Fe³⁺-cured Dopa-BS biofilms) were fixed by incubation with fixative solution containing 2% glutaraldehyde and 2% paraformaldehyde overnight at 4° C. The treated samples were then washed with a copious amount of ddH₂O. Samples were then dehydrated with an ethanol dehydration series (50%, 60%, 70%, 80%, 90% and 100% ethanol). The air-dried samples were then sputter-coated with gold for 10 seconds. SEM and EDS images were both taken from a JSM 7800 scanning electron microscope equipped with an Oxford X-max energy dispersive spectrometer. Results are presented in FIG. 23.

Attenuated Total Reflection Fourier Transform Infrared Spectroscopy (ATR-FTIR)

To confirm EPS-Fe³⁺ interaction in the cured biofilm samples, ATR-FTIR methodology was applied. To prepare corresponding samples for ATR-FTIR analysis, the bslA⁻ tasA⁻ eps⁻ and bslA⁻ tasA⁻ biofilms were first scraped from MSgg plates (both weighing 2 g) and then thoroughly resuspended in 20 mL ddH₂O until no visible aggregates were detected. The obtained biofilm suspensions were either added with or without 10 μL of 1M FeCl₃ solution. Both samples were then freeze-dried by a lyophilizer (LABCONCO) for 2 days. The dried samples were then tested using Fourier transform infrared spectrometer (PerkinElmer) with a range of wavenumber from 900 cm⁻¹ to 1800 cm⁻¹. Results are presented in FIG. 24.

Fluorescence Microscopy.

To verify the expression and secretion of the BslA-Mfp3Sp fusion protein, we first constructed a strain producing BslA-Mfp3Sp-Spytag (see above) that facilitates fluorescence labeling via Spytag/Spycatcher protein partner interactions [4]. The obtained biofilms containing bslA⁻/BslA-Mfp3Sp-Spytag were confirmed with green or red fluorescence when incubated with the freshly purified GFP-Spycatcher protein or mCherry-Spycatcher protein.

Two biofilms samples, each correspondingly containing expressed BslA-Mfp3Sp-Spytag and BslA-Mfp3Sp, were grown from 4 mL MSgg liquid medium and collected by centrifugation at 4000 g for 5 min. The biofilm samples were then incubated with 1 mL 1 mg/mL GFP-Spycatcher or mCherry-Spycatcher protein solutions for 1 h at room temperature. Afterwards, samples were washed 3 times with 1 mL PBS buffer through centrifugation (5000 g for 5 min) and resuspension.

To prepare samples for fluorescent imaging, 10 μL of resuspended sample solution was spotted onto a glass slide by a pipette and covered with a coverslip carefully. Fluorescent imaging was then carried out with a Zeiss Axio Imager 2 fluorescence microscope under identical laser power using the 488 nm exciting channel for green fluorescence detection.

Rheology Measurement.

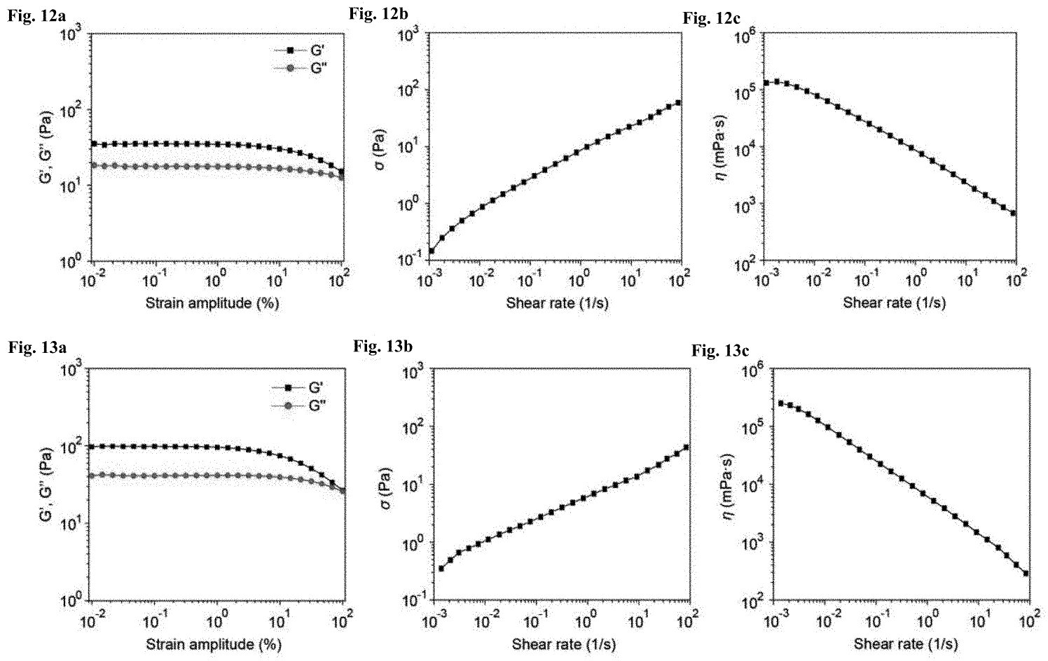

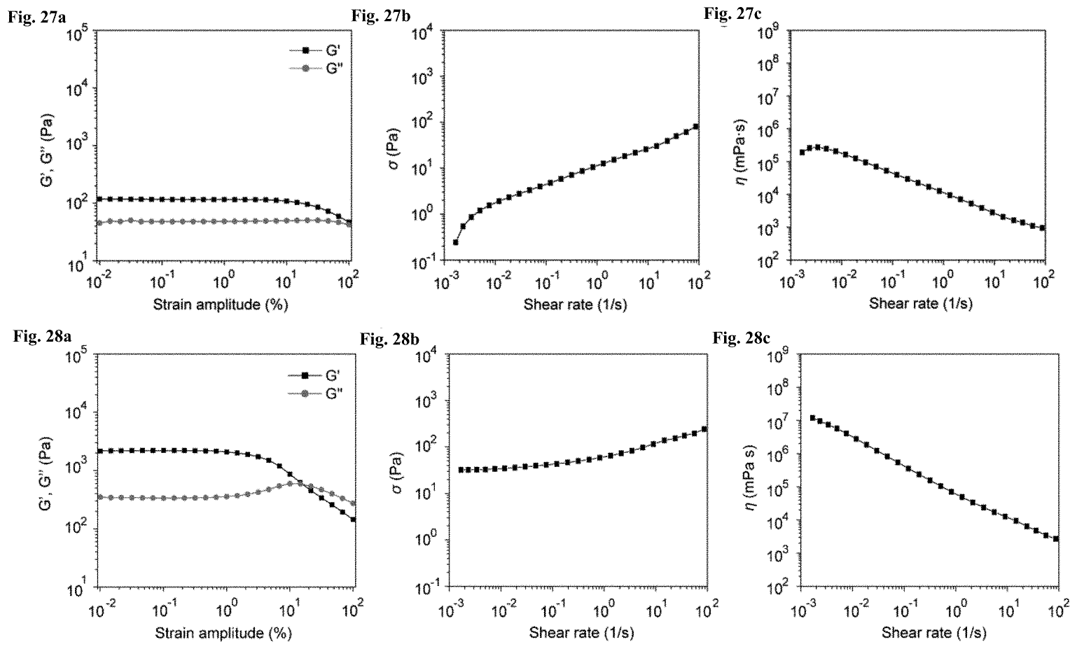

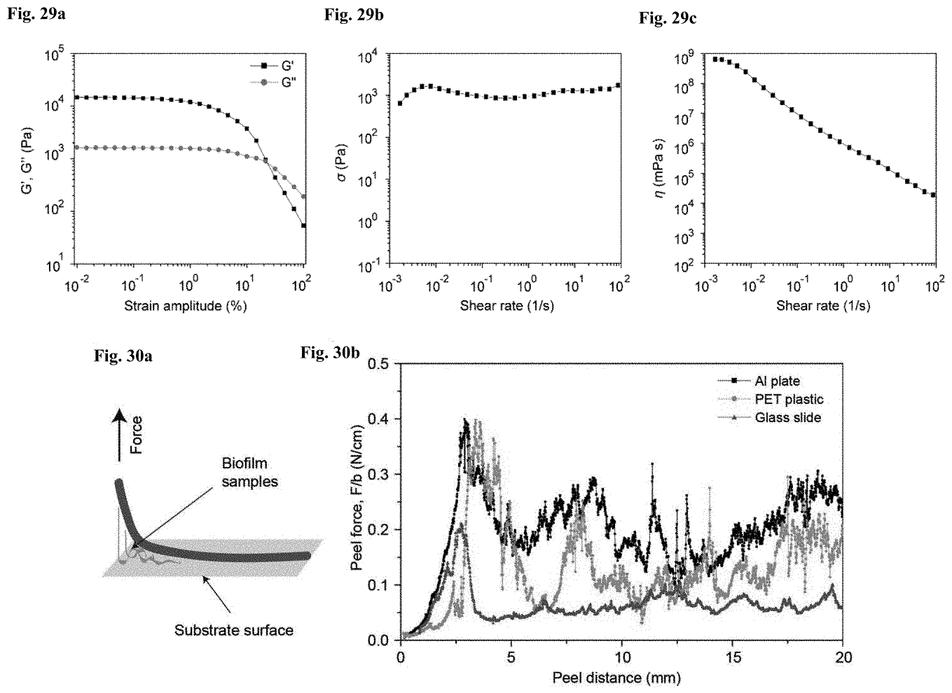

The viscoelastic properties of engineered cellular glues were assessed with a rheometer. Biofilm samples scraped from MSgg plates were directly applied for rheological tests except for the Fe³⁺ cured biofilm, which was prepared by mixing 5 μL, 1M FeCl₃ solution with 0.5 gram Dopa-BS biofilm. In a typical measurement, the biofilm sample was placed on the 25 mm diameter cone plate (101 pin gap) equipped with the strain-controlled rheometer (Anton paar MCR 302). To minimize the water evaporation of biofilm samples during tests, the measurements were carried out in a closed chamber containing pure water surrounding the cone plate. The storage modulus and loss modulus of biofilms were measured in a strain-controlled model test with strain amplitude ranging from 0.01% to 10% at a constant frequency of 10 rad/s. The stress and viscosity of biofilms were obtained in shear-controlled measurements at shear rates ranging from 0.001 to 100 l/s. The temperature was kept constant at 25° C. throughout the experiments with a Peltier thermoelectric device. Results are presented in FIGS. 11-13, FIG. 16, FIGS. 19-21, and FIGS. 27-29.

Lap Shear Measurement.

The adhesion properties of biofilm glues were evaluated using the lap shear test similar to the method described in previous studies [5]. Specifically, biofilm samples scraped from MSgg plate (weight at 0.2±0.02 g) were applied uniformly between two stainless steels sheets (10×60×0.05 mm) with a 1×1 cm overlapped area. Similarly, the metal ions-cured Dopa-BS biofilm samples were prepared by mixing 2 μL 1M CaCl₂ (pH 5.9), 1M MgCl₂ (pH 6.3), or 1 M FeCl₃ (pH 1.0) solution with 0.2 gram Dopa-BS biofilm and then applied to the sheets. The two sheets glued by the wet biofilm samples were then incubated for 2 h at 30° C. under 30% relative humidity level before measurement.

To assess the influence of relative humidity levels on adhesion properties of biofilm glues, the wet biofilm samples applied between the two sheets were incubated for 2 h at 30° C. under different humidity (30%, 50%, 70%, and 90%) before measurement.

To assess the adhesion performance of biofilm glues challenged under different detergent treatments, the cultured biofilms were first immersed into the detergent solution (0.1% m/v Sodium Dodecyl Sulfonate (SDS) or 8 M Urea solution) and incubated for 5 min. The samples were blotted off with the excessive solutions before they were applied as glues between the two sheets, followed by incubation at 30° C. under 30% humidity for 2 h.

To assess the adhesion performance of biofilm glues exposure to different concentrations of ion solutions (Ca²⁺, Mg²⁺, or Fe³⁺), the Dopa-BS biofilm samples (0.2 gram) were prepared by firstly mixing with 2 μL ion solutions and then directly placed and flattened on the stainless steel sheets, followed by incubation at 30° C. under 30% humidity for 2 h.

To assess the adhesion performance of biofilm glues on different substrate, the wild type, Tyr-BS, Dopa-BS, and Fe³⁺-cured Dopa-BS biofilms were applied on different substrates (polytetrafluoroethylene (PTFE), aluminum foil, and polyethylene terephthalate (PET)), with a 1×1 cm overlapped area as described above. The samples were then incubated at 30° C. under 30% humidity for 2 h before tests.

To assess the influence of pH on adhesion performance of metal ion-cured biofilm glues, the Dopa-BS biofilm samples were first immersed in solutions with different pH values (HCl (pH 1.0), NaCl (pH 7.0), NaOH (pH 12.0)) for 5 min before scraped off from MSgg plate. The metal ions-cured Dopa-BS biofilm samples were prepared by mixing 2 μL 1M CaCl₂, MgCl₂, or FeCl₃ solution with 0.2 gram Dopa-BS biofilm and then applied to stainless steel sheets as described above. The samples were then incubated at 30° C. under 30% humidity for 2 h before tests.

To assess the influence of harsh conditions on the adhesion capacity of the Fe³⁺-cured Dopa-BS biofilm glue, the Fe³⁺-cured Dopa-BS biofilm samples were applied on the substrate of terephthalate (PET) sheets with a 1×1 cm overlapped area as described above, followed by full immersion into different aqueous solutions (HCl (pH 3.0), NaOH (pH 12.0), or CH₃OH solution) at room temperature for 10 h.

After 2 h incubation, the glued stainless-steel sheets were taken out from the chamber and immediately anchored in a universal material testing machine (Instron 5966) equipped with a (maximum) 120 N mechanical sensor using two vertical (maximum) 100 N tensile clamps. The gap distance between two clamps was set at 5 cm for initial loading and the tensile speed was set at 5 mm/min during all measurements except for those shear speed tests of biofilm glues.

To assess the influence of shear speed on the adhesive capacities of biofilm glues, the Dopa-BS and Fe³⁺-cured Dopa-BS biofilms were applied to the stainless steel sheets with a 1×1 cm overlapped area as described above, followed by incubation at 30° C. under 30% humidity for 2 h. A wide range of shear speeds (0.1-100 mm/min) was conducted to measure the effect of shear speeds on the shear adhesive strength of Dopa-BS and Fe³⁺-cured Dopa-BS biofilm glues.

The ultimate shear adhesive strength of the biofilms was defined by a maximum shear-load force divided by the overlapped area where biofilms samples were applied. The experimental setup for typical force-distance curves and means by which corresponding adhesion strength data are obtained from the curves are presented in FIG. 14.

90° Degree Peel Measurement.

The 90° peel measurement was conducted to assess the peel adhesive capacity of the Dopa-BS biofilm glue on different surfaces of substrates following a similar peel test method as described in previous studies [6]. The Dopa-BS biofilms were painted uniformly between a non-woven tape and different substrates (aluminum plate, polyethylene terephthalate (PET) plastic, and glass slide) with an overlapped area of 2×1 cm² (length: 2 cm; width: 1 cm). The thickness of all biofilm samples is 50 μm. The measurement of the 90° peel test was performed using a universal material testing machine (Instron 5966) equipped with a ‘90 deg peel fixture dual column’ and the peel force was measured as function of peel distance at a constant peel rate of 5 mm/min. All Dopa-BS biofilm samples were incubated at 30° C. under 30% humidity for 2 h before measurement. The result is presented in FIG. 30.

Expression and Purification of the GFP-Spycatcher Protein.

The recombinant plasmid pET22b-GFP-Spycater was transformed into E. coli BL21 (DE3). The expression and purification of GFP-Spycatcher fusion protein were carried out following a previously described method [7]. Specifically, the strains were grown to OD600 0.5-1.0 in 1 L LB broth supplemented with 50 mg/mL ampicillin at 220 rpm 37° C. Protein expression was then induced with 0.5 mM IPTG at 16° C. for 12 h. Bacteria pellets were collected by centrifugation at 5000 g. Usually 5 gram cell pellets were harvested and resuspended in 50 mL lysis buffer (50 mM Tris-HCl, 500 mM NaCl, 20 mM β-mercaptoethanol (BME), pH 8.0). Lysozyme (final concentration of 0.2 mg/mL) and 0.1 mM PMSF were also added in the lysis buffer to inhibit protein degradation by enzymes. Lysates were then incubated on ice for 30 min, followed by sonication for 30 mM The insoluble portions of the lysates were removed through centrifugation at 30000 g for 1 hour, and the separated supernatants were incubated with 6 mL Ni-NTA resin (Clontech) for 30 mM at room temperature. Resin beads were then centrifuged at 200 g and washed with 300 mL of washing buffer (50 mM Tris-HCl, 500 mM NaCl, 20 mM imidazole, 20 mM BME, pH 7.2). The proteins were eluted from the gravity column with 20-30 mL elution buffer (50 mM Tris-HCl, 500 mM NaCl, 400 mM imidazole, 20 mM BME, pH 7.2) and stored at 4° C. for later use. Expression and purification of mCherry-Spycatcher protein followed the same protocol for GFP-Spycatcher protein described above.

Confirmation of tyrosinase expression under the biofilm forming conditions. The Dopa-BS strain harboring a tyrosinase gene was cultured in multiple culture dishes containing 4 mL liquid MSgg culture supplemented with 5 μg/mL chloramphenicol, 1 mM IPTG and 0.4 μg/mL CuSO₄ as described above. For protein purification, the biofilm solution was collected by centrifugation at 5000 g for 10 mM (scaling up the culture medium until 1 gram cell pellets were harvested) and resuspended in lysis buffer containing 50 mL protein extraction solution (8 M guanidine hydrochloride (GdnHCl), 300 mM NaCl, 50 mM K₂HPO₄/KH₂PO₄, pH 7.2). The insoluble portions of the lysates were removed through centrifugation at 30000 g for 1 hour, and the separated supernatants were incubated with 6 mL Ni-NTA resin (Clontech) for 30 mM at room temperature. Resin beads were then centrifuged at 200 g, and washed with 300 mL of washing buffer (50 mM Tris-HCl, 500 mM NaCl, 20 mM imidazole, pH 7.2). The proteins were eluted from the gravity column with 20-30 mL elute buffer (50 mM Tris-HCl, 500 mM NaCl, 400 mM imidazole, pH 7.2).

The purified tyrosinase protein solution was then detected by Western Blot assay. Specifically, protein solution was electrophoresed on 12% SDS-polyacrylamide gels and blotted onto polyvinylidene difluoride (PVDF) membranes using iBlot (Invitrogen). Western blots were probed by anti-His monoclonal mouse antibodies (LifeTein) at a dilution of 1:10,000. Secondary goat anti-mouse antibodies IgG conjugated to horseradish peroxidase (Sigma) were used at a dilution of 1:5,000. The blots were developed using the Pierce SuperSignal detection system and imaged using a Bio-Rad ChemiDoc MP system. The result is presented in FIG. 25a.

### METHODS REFERENCES

1. Spizizen, J., Proceedings of the National Academy of Sciences (1958) 44 (10), 1072
2. Wang, X. Y., et al., Adv Mater (2018) 30 (16)
3. Zhong, C., et al., Nat Nanotechnol (2014) 9 (10), 858
4. Keeble, A. H., et al., Angew Chem Int Edit (2017) 56 (52), 16521
5. Cha, H. J., et al., Biofouling (2009) 25 (2), 99
6. McGuiggan, P. M., et al., Int J Adhes Adhes (2008) 28 (4-5), 185
7. Wang, S., et al., Plos Comput Biol (2017) 13 (1)
8. Cagnasso, M., et al., Colloid Surface B (2010) 76 (2), 456

1. A fusion protein comprising a Bacillus biofilm-surface layer protein A (BslA) and a mussel foot protein 3S (Mfp3S) or coacervation inducing peptide thereof.

2. The fusion protein of claim 1 wherein the BslA is (B. subtilis), Ba_BslA (B. amyloliquefaciens), Bl_BslA (B. licheniformis), or Bp_BslA (B. pumilus).

3. The fusion protein of claim 1, wherein the peptide is Mfp3Sp.

4. A glue composition comprising an engineered Bacillus biofilm comprising:
a) a first fusion protein of claim 1;
b) a second fusion protein comprising an amyloid protein functionalized with a marine adhesion protein; and
c) exopolysaccharide (EPS).

5. A glue composition comprising an engineered Bacillus biofilm comprising:
a) a first fusion protein of claim 2;
b) a second fusion protein comprising an amyloid protein functionalized with a marine adhesion protein; and
c) exopolysaccharide (EPS).

6. A glue composition comprising an engineered Bacillus biofilm comprising:
a) a first fusion protein of claim 3;
b) a second fusion protein comprising an amyloid protein functionalized with a marine adhesion protein; and
c) exopolysaccharide (EPS).

7. The composition of claim 4 wherein the Bacillus is Bacillus subtilis, Bacillus amyloliquefaciens, Bacillus licheniformis, or Bacillus pumilus.

8. The composition of claim 4 wherein the amyloid protein is TasA (B. subtilis), CsgA amyloid (E. coli), PSMs amyloid (S. aureus), or FapC (Pseudomonas spp.).

9. The composition of claim 4 wherein the marine adhesion protein is muscle foot adhesive proteinMfp5, Mefp3, Mcfp3, or Mcfp5.

10. The composition of claim 4 wherein:
the amyloid protein is TasA (B. subtilis), CsgA amyloid (E. coli), PSMs amyloid (S. aureus), or FapC (Pseudomonas spp.); and
the marine adhesion protein is muscle foot adhesive proteinMfp5, Mefp3, Mcfp3, or Mcfp5.

11. The composition of claim 5 wherein:
the amyloid protein is TasA (B. subtilis), CsgA amyloid (E. coli), PSMs amyloid (S. aureus), or FapC (Pseudomonas spp.); and
the marine adhesion protein is muscle foot adhesive proteinMfp5, Mefp3, Mcfp3, or Mcfp5.

12. The composition of claim 6 wherein:
the amyloid protein is TasA (B. subtilis), CsgA amyloid (E. coli), PSMs amyloid (S. aureus), or FapC (Pseudomonas spp.); and
the marine adhesion protein is muscle foot adhesive proteinMfp5, Mefp3, Mcfp3, or Mcfp5.

13. The composition of claim 4 wherein one or both of the fusion proteins comprise dihydroxyphenylalanine (DOPA) residues.

14. The composition of claim 4 cured with one or more metal ions (e.g. Ca²⁺, Mg²⁺, Fe³⁺).

15. The composition of claim 4 comprising living, growing Bacillus microbes.

16. The composition of claim 5 comprising living, growing Bacillus microbes.

17. The composition of claim 6 comprising living, growing Bacillus microbes.

18. The composition of claim 4 comprising viscoelasticity sufficient to be injectable into crevices or holes, and/or self-regenerative ability to maintain adhesive strength after multiple generations/passages.

19. A composition comprising an engineered Bacillus microbe comprising the fusion protein of claim 1, and:
a) a first recombinant gene encoding the fusion protein; and
b) a second recombinant gene encoding a second fusion protein comprising an amyloid protein and a marine adhesion protein,
optionally further comprising a recombinant, heterologous, inducible gene encoding a tyrosinase which catalyzes the hydroxylation of tyrosine residues to form Dopa on one or both of the fusion proteins.

20. A method of making a glue composition comprising growing the microbe of claim 83 under conditions wherein a biofilm comprising the fusion proteins is formed.
